(19) United States
(12) Patent Application Publication
(10) Pub. No.: US 2008/0065153 A1
(43) Pub. Date: Mar. 13, 2008

(54) Surgical staple

(75) Inventors: Randall N. Allard, Germantown, TN (US); Larry T. McBride, Ripley, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC, INC., Warsaw, IN (US)

(21) Appl. No.: 11/518,059

(22) Filed: Sep. 8, 2006

(51) Int. Cl. A61B 17/08

(52) U.S. Cl. 606/219

(57) ABSTRACT

A surgical staple is disclosed and can include a base and at least one tine that can extend from the base. Further, at least one tissue engagement structure can be incorporated into the at least one tine. The at least one tissue engagement structure can be moved between a retracted configuration and an extended configuration.

### FIELD OF THE DISCLOSURE

The present disclosure relates generally to surgical staples. More specifically, the present disclosure relates to surgical staples having various features configured to engage bone, flesh, or a combination thereof.

### BACKGROUND

Surgical staples can be used to close surgical wounds and other wounds. Further, surgical staples can be used to treat certain spinal deformities, such as scoliosis. Some surgical staples can be made from shape memory metal alloys. A surgical staple made from a shape memory metal alloy can be deformed from an original shape and installed in a patient. Then, heat can be applied to the surgical staple to return the deformed staple to the original shape.

### BRIEF DESCRIPTION OF THE DRAWINGS

FIG. 1 is a perspective view of a first embodiment of a two-tine surgical staple;

FIG. 2 is a plan view of the first embodiment of the two-tine surgical staple in an open configuration;

FIG. 3 is a plan view of the first embodiment of the two-tine surgical staple in a closed configuration;

FIG. 4 is a perspective view of a second embodiment of a two-tine surgical staple;

FIG. 5 is a plan view of the second embodiment of the two-tine surgical staple in a closed configuration;

FIG. 6 is a plan view of the second embodiment of the two-tine surgical staple in an open configuration;

FIG. 7 is a plan view of a third embodiment of a two-tine surgical staple;

FIG. 8 is a plan view of a first tine of the third embodiment of the two-tine surgical staple in a retracted configuration;

FIG. 9 is a plan view of the first tine of the third embodiment of the two-tine surgical staple in an extended configuration;

FIG. 10 is a plan view of a second tine of the third embodiment of the two-tine surgical staple in a retracted configuration;

FIG. 11 is a plan view of the second tine of the third embodiment of the two-tine surgical staple in an extended configuration;

FIG. 12 is a plan view of a fourth embodiment of a two-tine surgical staple;

FIG. 13 is a plan view of a first tine of the fourth embodiment of the two-tine surgical staple in a retracted configuration;

FIG. 14 is a plan view of the first tine of the fourth embodiment of the two-tine surgical staple in an extended configuration;

FIG. 15 is a plan view of a second tine of the fourth embodiment of the two-tine surgical staple in a retracted configuration;

FIG. 16 is a plan view of the second tine of the fourth embodiment of the two-tine surgical staple in an extended configuration;

FIG. 17 is a plan view of a fifth embodiment of a two-tine surgical staple;

FIG. 18 is a plan view of a first tine of the fifth embodiment of the two-tine surgical staple in a retracted configuration;

FIG. 19 is a plan view of the first tine of the fifth embodiment of the two-tine surgical staple in an extended configuration;

FIG. 20 is a plan view of a second tine of the fifth embodiment of the two-tine surgical staple in a retracted configuration;

FIG. 21 is a plan view of the second tine of the fifth embodiment of the two-tine surgical staple in an extended configuration;

FIG. 22 is a plan view of a sixth embodiment of a two-tine surgical staple;

FIG. 23 is a plan view of a first tine of the sixth embodiment of the two-tine surgical staple in a retracted configuration;

FIG. 24 is a plan view of the first tine of the sixth embodiment of the two-tine surgical staple in an extended configuration;

FIG. 25 is a plan view of a second tine of the sixth embodiment of the two-tine surgical staple in a retracted configuration;

FIG. 26 is a plan view of the second tine of the sixth embodiment of the two-tine surgical staple in an extended configuration;

FIG. 27 is a plan view of a seventh embodiment of a two-tine surgical staple;

FIG. 28 is a plan view of a first tine of the seventh embodiment of the two-tine surgical staple in a retracted configuration;

FIG. 29 is a plan view of the first tine of the seventh embodiment of the two-tine surgical staple in an extended configuration;

FIG. 30 is a plan view of a second tine of the seventh embodiment of the two-tine surgical staple in a retracted configuration;

FIG. 31 is a plan view of the second tine of the seventh embodiment of the two-tine surgical staple in an extended configuration;

FIG. 32 is a plan view of a eighth embodiment of a two-tine surgical staple;

FIG. 33 is a plan view of a first tine of the eighth embodiment of the two-tine surgical staple in a retracted configuration;

FIG. 34 is a plan view of the first tine of the eighth embodiment of the two-tine surgical staple in an extended configuration;

FIG. 35 is a plan view of a second tine of the eighth embodiment of the two-tine surgical staple in a retracted configuration;

FIG. 36 is a plan view of the second tine of the eighth embodiment of the two-tine surgical staple in an extended configuration;

FIG. 37 is a plan view of a ninth embodiment of a two-tine surgical staple;

FIG. 38 is a plan view of a first tine of the ninth embodiment of the two-tine surgical staple in a retracted configuration;

FIG. 39 is a plan view of the first tine of the ninth embodiment of the two-tine surgical staple in an extended configuration;

FIG. 40 is a plan view of a second tine of the ninth embodiment of the two-tine surgical staple in a retracted configuration;

FIG. 41 is a plan view of the second tine of the ninth embodiment of the two-tine surgical staple in an extended configuration;

FIG. 42 is a perspective view of a first embodiment of a three-tine surgical staple;

FIG. 43 is a first plan view of the first embodiment of the three-tine surgical staple;

FIG. 44 is a second plan view of the first embodiment of the three-tine surgical staple;

FIG. 45 is a third plan view of the first embodiment of the three-tine surgical staple;

FIG. 46 is a fourth plan view of the first embodiment of the three-tine surgical staple;

FIG. 47 is a fifth plan view of the first embodiment of the three-tine surgical staple;

FIG. 48 is a sixth plan view of the first embodiment of the three-tine surgical staple;

FIG. 49 is a perspective view of a second embodiment of a three-tine surgical staple;

FIG. 50 is a first plan view of the second embodiment of the three-tine surgical staple;

FIG. 51 is a second plan view of the second embodiment of the three-tine surgical staple;

FIG. 52 is a third plan view of the second embodiment of the three-tine surgical staple;

FIG. 53 is a fourth plan view of the second embodiment of the three-tine surgical staple;

FIG. 54 is a fifth plan view of the second embodiment of the three-tine surgical staple;

FIG. 55 is a sixth plan view of the second embodiment of the three-tine surgical staple;

FIG. 56 is a perspective view of a first embodiment of a four-tine surgical staple;

FIG. 57 is a first plan view of the first embodiment of the four-tine surgical staple in a closed configuration;

FIG. 58 is a second plan view of the first embodiment of the four-tine surgical staple in a closed configuration;

FIG. 59 is a third plan view of the first embodiment of the four-tine surgical staple in an open configuration;

FIG. 60 is a perspective view of a second embodiment of a four-tine surgical staple;

FIG. 61 is a first plan view of the second embodiment of the four-tine surgical staple;

FIG. 62 is a second plan view of the second embodiment of the four-tine surgical staple;

FIG. 63 is a third plan view of the second embodiment of the four-tine surgical staple;

FIG. 64 is a fourth plan view of the second embodiment of the four-tine surgical staple;

FIG. 65 is a fifth plan view of the second embodiment of the four-tine surgical staple;

FIG. 66 is a sixth plan view of the second embodiment of the four-tine surgical staple;

FIG. 67 is a perspective view of a third embodiment of a four-tine surgical staple;

FIG. 68 is a first plan view of the third embodiment of the four-tine surgical staple;

FIG. 69 is a second plan view of the third embodiment of the four-tine surgical staple;

FIG. 70 is a third plan view of the third embodiment of the four-tine surgical staple;

FIG. 71 is a fourth plan view of the third embodiment of the four-tine surgical staple;

FIG. 72 is a fifth plan view of the third embodiment of the four-tine surgical staple;

FIG. 73 is a flow chart illustrating a method of using a surgical staple.

### DETAILED DESCRIPTION OF THE DRAWINGS

A surgical staple is disclosed and can include a base and at least one tine that can extend from the base. Further, at least one tissue engagement structure can be incorporated into the at least one tine. The at least one tissue engagement structure can be moved between a retracted configuration and an extended configuration.

In another embodiment, a surgical staple is disclosed and can include a base. A first tine can extend from the base. Further, a first tissue engagement structure and a second tissue engagement structure can be incorporated into the first tine. A second tine can extend from the base opposite the first tine. Also, a first tissue engagement structure and a second tissue engagement structure can be incorporated into the second tine. Each tissue engagement structure can be moved between a retracted configuration and an extended configuration.

In yet another embodiment, a surgical staple is disclosed and can include a base. A first tine can extend from the base. Further, a first tissue engagement structure and a second tissue engagement structure can be incorporated into the first tine. A second tine can extend from the base adjacent to the first tine. Also, a first tissue engagement structure and a second tissue engagement structure can be incorporated into the second tine. A third tine can extend from the base opposite the first tine and the second tine. A first tissue engagement structure and a second tissue engagement structure can be incorporated into the third tine. Each tissue engagement structure can be moved between a retracted configuration and an extended configuration.

In still another embodiment, a surgical staple is disclosed and can include a base. A first tine can extend from the base. Further, a first tissue engagement structure and a second tissue engagement structure can be incorporated into the first tine. A second tine can extend from the base adjacent to the first tine. Also, a first tissue engagement structure and a second tissue engagement structure can be incorporated into the second tine. A third tine can extend from the base opposite the first tine and the second tine. A first tissue engagement structure and a second tissue engagement structure can be incorporated into the third tine. A fourth tine can extend from the base adjacent to the third tine. A first tissue engagement structure and a second tissue engagement structure can be incorporated into the fourth tine. Each tissue engagement structure can be moved between a retracted configuration and an extended configuration.

### Description of a First Embodiment of a Two-Tine Surgical Staple

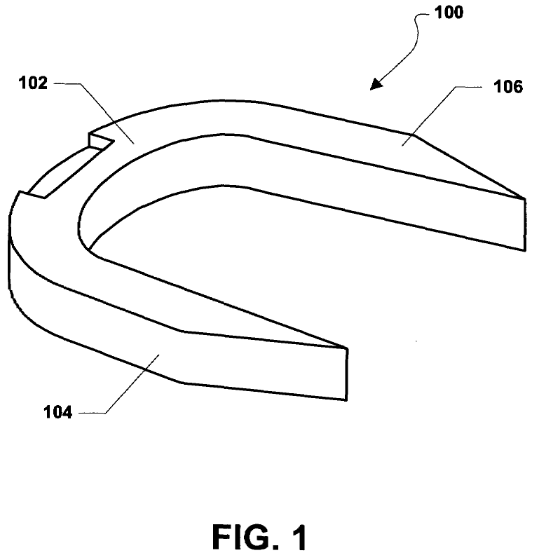

Referring initially to FIG. 1 through FIG. 3, a first embodiment of a two-tine surgical staple is shown and is generally designated 100. As shown, the two-tine surgical staple 100 can be generally U-shaped and can include a base 102. A first tine 104 and a second tine 106 can extend from the base 102.

As depicted, the first tine 104 can include a proximal end 110 and a distal end 112. Moreover, the first tine 104 can include an inner surface 114 and an outer surface 116. In a particular embodiment, the inner surface 114 of the first tine 104 can be relatively longer than the outer surface 116 of the first tine 104. As such, the distal end 112 of the first tine 104 can be angled from the inner surface 114 to the outer surface 116 and this angled portion can face outward relative to the surgical staple 100.

The second tine 106 can include a proximal end 120 and a distal end 122. Moreover, the second tine 106 can include an inner surface 124 and an outer surface 126. In a particular embodiment, the inner surface 124 of the second tine 106 can be relatively longer than the outer surface 126 of the second tine 106. As such, the distal end 122 of the second tine 106 can be angled from the inner surface 124 to the outer surface 126 and this angled portion can face outward relative to the surgical staple 100.

FIG. 1 through FIG. 3 also indicate that the base 102 of the surgical staple 100 can be formed with a slot 130. In a particular embodiment, the slot 130 can facilitate retrieval and insertion of the surgical staple 100.

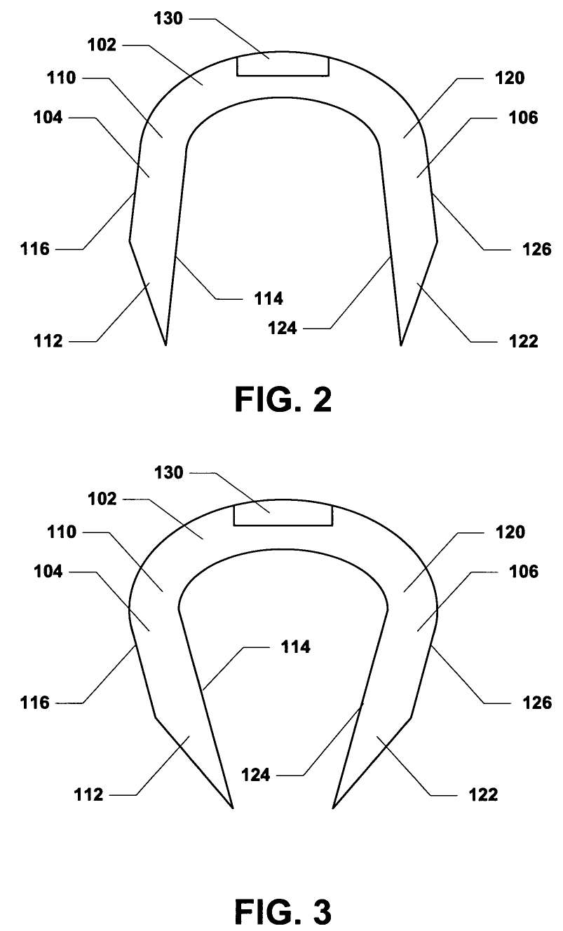

The surgical staple 100 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. As shown in FIG. 2 and FIG. 3, the surgical staple 100 can be moved between an open configuration, shown in FIG. 2, and a closed configuration, shown in FIG. 3. In a particular embodiment, the surgical staple 100 can be moved from the open configuration to the closed configuration by applying heat to the surgical staple 100.

### Description of a Second Embodiment of a Two-Time Surgical Staple

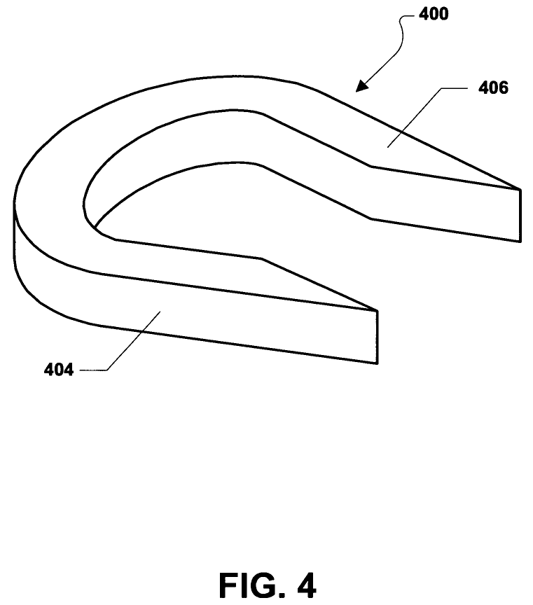

Referring to FIG. 4 through FIG. 6, a second embodiment of a two-tine surgical staple is shown and is generally designated 400. As shown, the two-tine surgical staple 400 can be generally U-shaped and can include a base 402. A first tine 404 and a second tine 406 can extend from the base 402.

As depicted, the first tine 404 can include a proximal end 410 and a distal end 412. Moreover, the first tine 404 can include an inner surface 414 and an outer surface 416. In a particular embodiment, the inner surface 414 of the first tine 404 can be relatively shorter than the outer surface 416 of the first tine 404. As such, the distal end 412 of the first tine 404 can be angled from the inner surface 414 to the outer surface 416 and this angled portion can face inward relative to the surgical staple 400.

The second tine 406 can include a proximal end 420 and a distal end 422. Moreover, the second tine 406 can include an inner surface 424 and an outer surface 426. In a particular embodiment, the inner surface 424 of the second tine 406 can be relatively shorter than the outer surface 426 of the second tine 406. As such, the distal end 422 of the second tine 406 can be angled from the inner surface 424 to the outer surface 426 and this angled portion can face inward relative to the surgical staple 400.

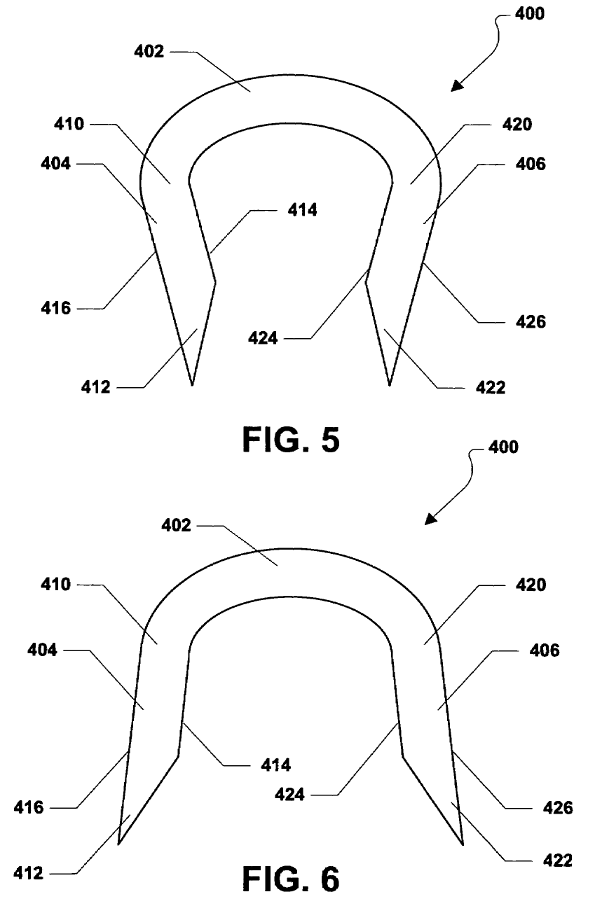

The surgical staple 400 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. As shown in FIG. 5 and FIG. 6, the surgical staple 400 can be moved between a closed configuration, shown in FIG. 5, and an open configuration, shown in FIG. 6. In a particular embodiment, the surgical staple 400 can be moved from the open configuration to the closed configuration by applying heat to the surgical staple 400.

### Description of a Third Embodiment of a Two-Tine Surgical Staple

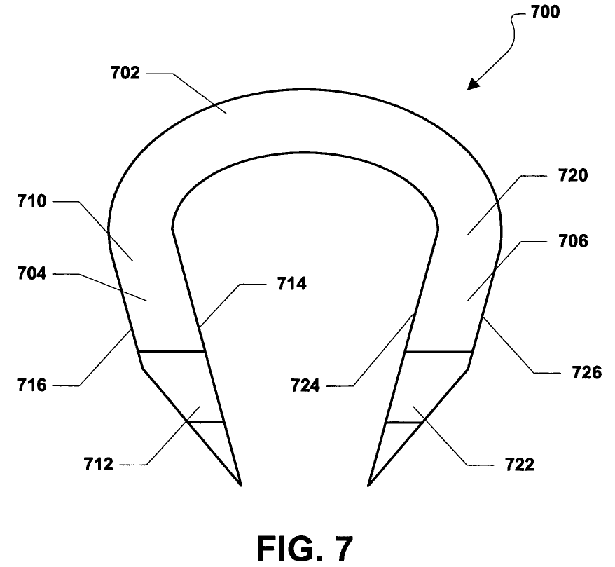

Referring to FIG. 7 through FIG. 11, a third embodiment of a two-tine surgical staple is shown and is generally designated 700. As shown, the two-tine surgical staple 700 can be generally U-shaped and can include a base 702. A first tine 704 and a second tine 706 can extend from the base 702.

As depicted, the first tine 704 can include a proximal end 710 and a distal end 712. Moreover, the first tine 704 can include an inner surface 714 and an outer surface 716. In a particular embodiment, the inner surface 714 of the first tine 704 can be relatively longer than the outer surface 716 of the first tine 704. As such, the distal end 712 of the first tine 704 can be angled from the inner surface 714 to the outer surface 716 and this angled portion can face outward relative to the surgical staple 700.

The second tine 706 can include a proximal end 720 and a distal end 722. Moreover, the second tine 706 can include an inner surface 724 and an outer surface 726. In a particular embodiment, the inner surface 724 of the second tine 706 can be relatively longer than the outer surface 726 of the second tine 706. As such, the distal end 722 of the second tine 706 can be angled from the inner surface 724 to the outer surface 726 and this angled portion can face outward relative to the surgical staple 700.

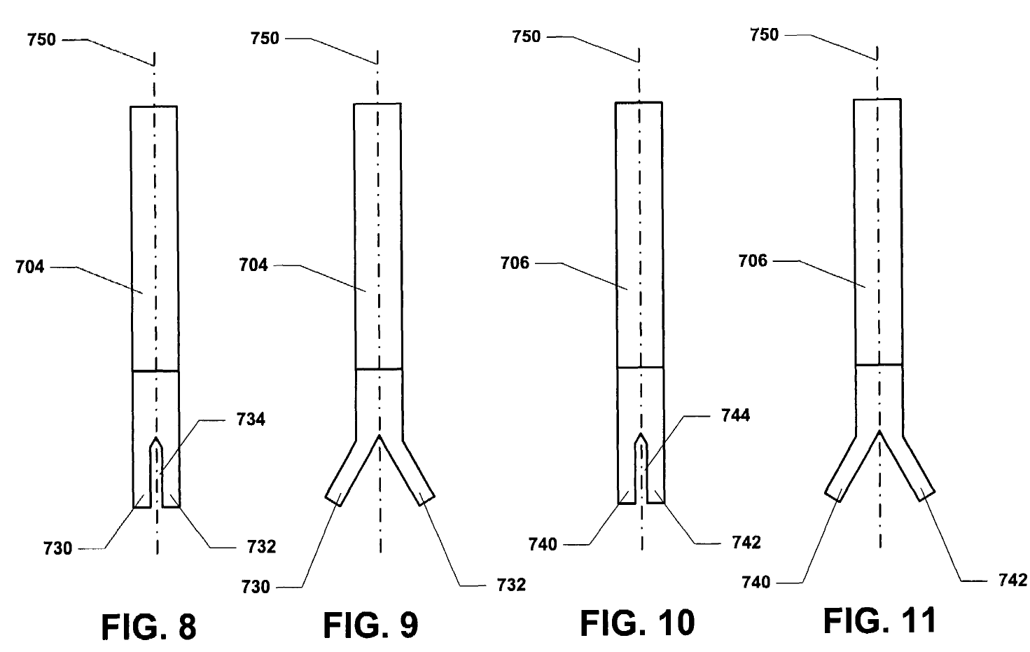

As illustrated in FIG. 8 and FIG. 9, the first tine 704 can include a first tissue engagement structure 730 and a second tissue engagement structure 732 that can extend from the distal end 712 of the first tine 704. Further, the first tissue engagement structure 730 and the second tissue engagement structure 732 can be separated by a void 734. The second tine 706 can include a first tissue engagement structure 740 and a second tissue engagement structure 742 that can extend from the distal end 722 of the second tine 706. The first tissue engagement structure 740 and the second tissue engagement structure 742 can be separated by a void 744. As shown, the tissue engagement structures 730, 732, 740, 742 can be substantially straight arms that can extend from, or be otherwise formed in, the distal end 712, 722 of each tine 704, 706.

The surgical staple 700 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 700 can be moved between a closed configuration and an open configuration or between an open configuration and a closed configuration, described above.

Further, the tissue engagement structures 730, 732, 740, 742 of each tine 704, 706 can be moved between a retracted configuration, shown in FIG. 8 and FIG. 10, and an extended configuration, shown in FIG. 9 and FIG. 11. In the retracted configuration, the tissue engagement structures 730, 732, 740, 742 can be substantially parallel to a longitudinal axis 750 of the surgical staple 700. Also, in the retracted configuration, the distal end 712, 722 of each tine 704, 706 can be generally fork-shaped due to the configuration of the tissue engagement structures 730, 732, 740, 742.

In the extended configuration, the tissue engagement structures 730, 732, 740, 742 can be angled with respect to the longitudinal axis 750 of the surgical staple 700. Further, in the extended configuration, each pair of adjacent tissue engagement structures can form a generally V-shaped structure that opens away from the base 702 of the surgical staple 700. Also, in the extended configuration the tissue engagement structures 730, 732, 740, 742 can engage tissue, e.g., bone or flesh, and substantially minimize the likelihood that the surgical staple 700 will be prematurely withdrawn from the tissue.

In a particular embodiment, the tissue engagement structures 730, 732, 740, 742 of the surgical staple 700 can be moved from the retracted configuration to the extended configuration by applying heat to the surgical staple 700.

### Description of a Fourth Embodiment of a Two-Tine Surgical Staple

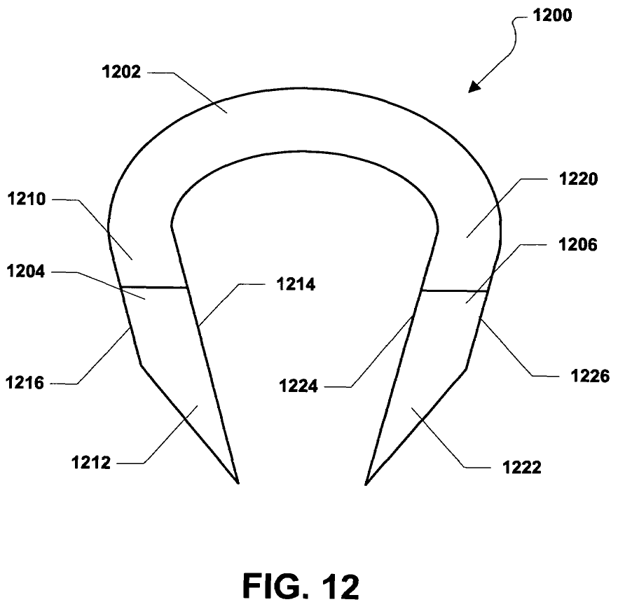

Referring to FIG. 12 through FIG. 16, a fourth embodiment of a two-tine surgical staple is shown and is generally designated 1200. As shown, the two-tine surgical staple 1200 can be generally U-shaped and can include a base 1202. A first tine 1204 and a second tine 1206 can extend from the base 1202.

As depicted, the first tine 1204 can include a proximal end 1210 and a distal end 1212. Moreover, the first tine 1204 can include an inner surface 1214 and an outer surface 1216. In a particular embodiment, the inner surface 1214 of the first tine 1204 can be relatively longer than the outer surface 1216 of the first tine 1204. As such, the distal end 1212 of the first tine 1204 can be angled from the inner surface 1214 to the outer surface 1216 and this angled portion can face outward relative to the surgical staple 1200.

The second tine 1206 can include a proximal end 1220 and a distal end 1222. Moreover, the second tine 1206 can include an inner surface 1224 and an outer surface 1226. In a particular embodiment, the inner surface 1224 of the second tine 1206 can be relatively longer than the outer surface 1226 of the second tine 1206. As such, the distal end 1222 of the second tine 1206 can be angled from the inner surface 1224 to the outer surface 1226 and this angled portion can face outward relative to the surgical staple 1200.

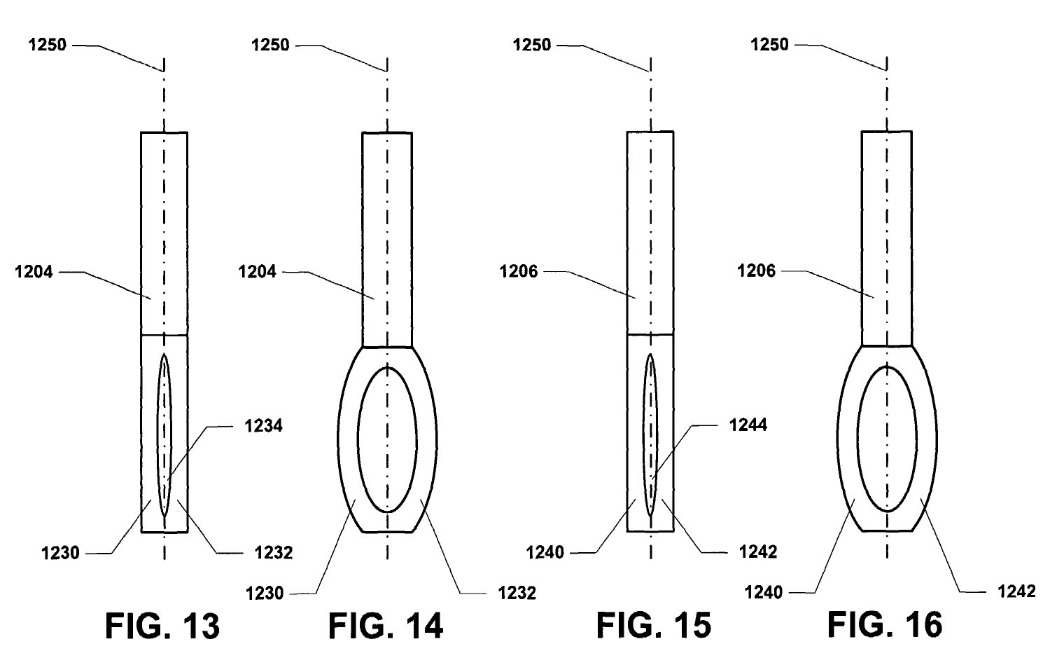

As illustrated in FIG. 13 and FIG. 14, the first tine 1204 can include a first tissue engagement structure 1230 and a second tissue engagement structure 1232 that can be incorporated in the distal end 1212 of the first tine 1204. Further, the first tissue engagement structure 1230 and the second tissue engagement structure 1232 can be separated by a void 1234. Moreover, as shown in FIG. 15 and FIG. 16, the second tine 1206 can include a first tissue engagement structure 1240 and a second tissue engagement structure 1242 that can be incorporated in the distal end 1222 of the second tine 1206. The first tissue engagement structure 1240 and the second tissue engagement structure 1242 can be separated by a void 1244. As shown in FIG. 13 and FIG. 15, the tissue engagement structures 1230, 1232, 1240, 1242 can be substantially straight arms formed in the distal end 1212, 1222 of each tine 1204, 1206.

The surgical staple 1200 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 1200 can be moved between a closed configuration and an open configuration or between an open configuration and a closed configuration, described above.

Further, the tissue engagement structures 1230, 1232, 1240, 1242 of each tine 1204, 1206 can be moved between a retracted configuration, shown in FIG. 13 and FIG. 15, and an extended configuration, shown in FIG. 14 and FIG. 16. In the retracted configuration, the tissue engagement structures 1230, 1232, 1240, 1242 can be substantially parallel to a longitudinal axis 1250 of the surgical staple 1200. In the extended configuration, the tissue engagement structures 1230, 1232, 1240, 1242 can be bowed or otherwise bent. Further, in the extend configuration, each pair of tissue engagement structures 1230, 1232 and 1240, 1242 can form a generally elliptical structure at the distal end 1212, 1222 of each tine 1204, 1206. Also, in the extended configuration the tissue engagement structures 1230, 1232, 1240, 1242 can engage tissue, e.g., bone or flesh, and substantially minimize the likelihood that the surgical staple 1200 will be prematurely withdrawn from the tissue.

In a particular embodiment, the tissue engagement structures 1230, 1232, 1240, 1242 of the surgical staple 1200 can be moved from the retracted configuration to the extended configuration by applying heat to the surgical staple 1200.

### Description of a Fifth Embodiment of a Two-Tine Surgical Staple

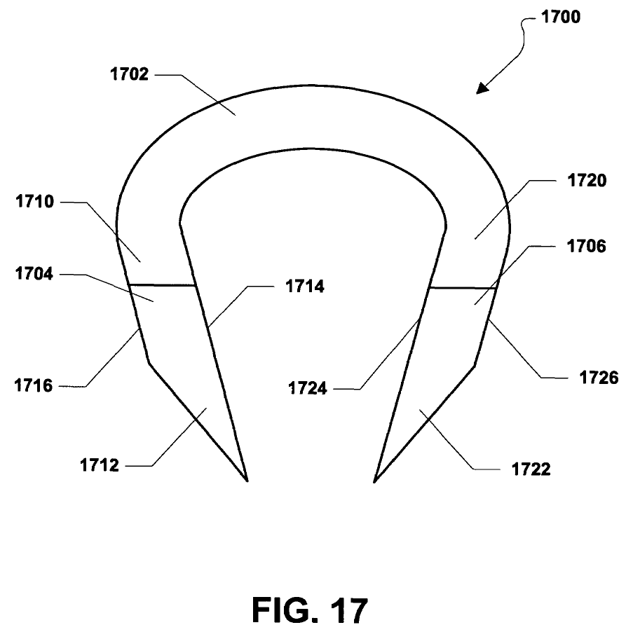

Referring to FIG. 17 through FIG. 21, a fifth embodiment of a two-tine surgical staple is shown and is generally designated 1700. As shown, the two-tine surgical staple 1700 can be generally U-shaped and can include a base 1702. A first tine 1704 and a second tine 1706 can extend from the base 1702.

As depicted, the first tine 1704 can include a proximal end 1710 and a distal end 1712. Moreover, the first tine 1704 can include an inner surface 1714 and an outer surface 1716. In a particular embodiment, the inner surface 1714 of the first tine 1704 can be relatively longer than the outer surface 1716 of the first tine 1704. As such, the distal end 1712 of the first tine 1704 can be angled from the inner surface 1714 to the outer surface 1716 and this angled portion can face outward relative to the surgical staple 1700.

The second tine 1706 can include a proximal end 1720 and a distal end 1722. Moreover, the second tine 1706 can include an inner surface 1724 and an outer surface 1726. In a particular embodiment, the inner surface 1724 of the second tine 1706 can be relatively longer than the outer surface 1726 of the second tine 1706. As such, the distal end 1722 of the second tine 1706 can be angled from the inner surface 1724 to the outer surface 1726 and this angled portion can face outward relative to the surgical staple 1700.

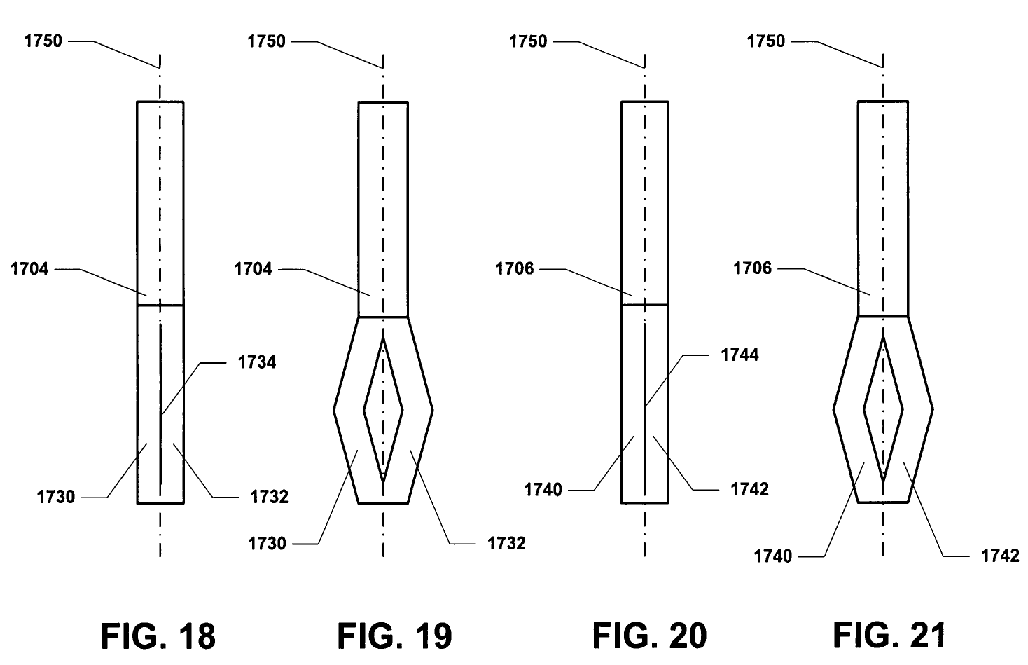

As illustrated in FIG. 18 and FIG. 19, the first tine 1704 can include a first tissue engagement structure 1730 and a second tissue engagement structure 1732 that can be incorporated in the distal end 1712 of the first tine 1704. Moreover, as shown in FIG. 20 and FIG. 21, the second tine 1706 can include a first tissue engagement structure 1740 and a second tissue engagement structure 1742 that can be incorporated in the distal end 1722 of the second tine 1706. As shown in FIG. 18 and FIG. 20, the tissue engagement structures 1730, 1732 of the first tine 1704 can be substantially straight arms that can be separated by a slit 1734 formed in the distal end 1712 of the first tine 1704. Also, the tissue engagement structures 1740, 1742 of the second tine 1706 can be substantially straight arms that can separated by a slit 1744 formed in the distal end 1712, 1722 of each tine 1704, 1706.

The surgical staple 1700 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 1700 can be moved between a closed configuration and an open configuration or between an open configuration and a closed configuration, described above.

Further, the tissue engagement structures 1730, 1732, 1740, 1742 of each tine 1704, 1706 can be moved between a retracted configuration, shown in FIG. 18 and FIG. 20, and an extended configuration, shown in FIG. 19 and FIG. 20. In the retracted configuration, the tissue engagement structures 1730, 1732, 1740, 1742 can be substantially parallel to a longitudinal axis 1750 of the surgical staple 1700.

In the extended configuration, the tissue engagement structures 1730, 1732, 1740, 1742 can be slightly folded at a midpoint of each tissue engagement structure 1730, 1732, 1740, 1742. Also, in the extended configuration, the slit 1734, 1744 in the distal end 1712, 1722 of each tine 1704, 1706 can open and form a generally diamond-shaped, parallelogrammical opening in the distal end 1712, 1722 of each tine 1704, 1706. Further, in the extend configuration, each pair of tissue engagement structures 1730, 1732 and 1740, 1742 can form a generally diamond-shaped, parallelogrammical structure at the distal end 1712, 1722 of each tine 1704, 1706. Also, in the extended configuration the tissue engagement structures 1730, 1732, 1740, 1742 can engage tissue, e.g., bone or flesh, and substantially minimize the likelihood that the surgical staple 1700 will be prematurely withdrawn from the tissue.

In a particular embodiment, the tissue engagement structures 1730, 1732, 1740, 1742 of the surgical staple 1700 can be moved from the retracted configuration to the extended configuration by applying heat to the surgical staple 1700.

### Description of a Sixth Embodiment of a Two-Tine Surgical Staple

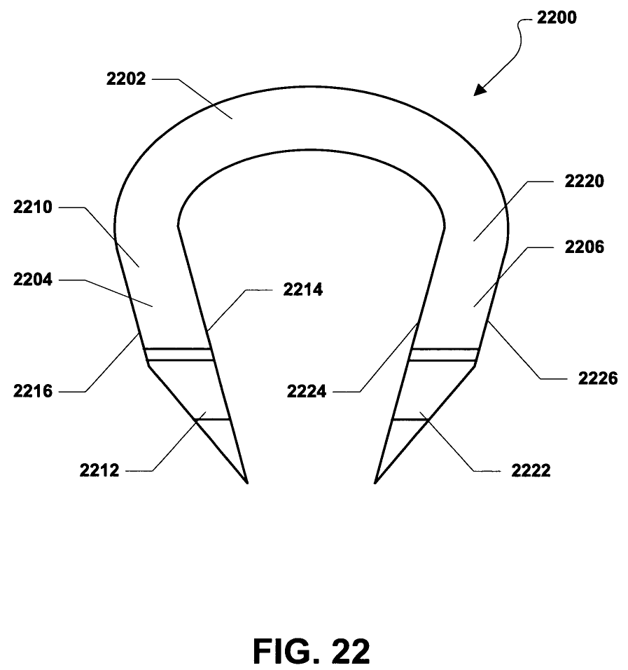

Referring to FIG. 22 through FIG. 26, a sixth embodiment of a two-tine surgical staple is shown and is generally designated 2200. As shown, the two-tine surgical staple 2200 can be generally U-shaped and can include a base 2202. A first tine 2204 and a second tine 2206 can extend from the base 2202.

As depicted, the first tine 2204 can include a proximal end 2210 and a distal end 2212. Moreover, the first tine 2204 can include an inner surface 2214 and an outer surface 2216. In a particular embodiment, the inner surface 2214 of the first tine 2204 can be relatively longer than the outer surface 2216 of the first tine 2204. As such, the distal end 2212 of the first tine 2204 can be angled from the inner surface 2214 to the outer surface 2216 and this angled portion can face outward relative to the surgical staple 2200.

The second tine 2206 can include a proximal end 2220 and a distal end 2222. Moreover, the second tine 2206 can include an inner surface 2224 and an outer surface 2226. In a particular embodiment, the inner surface 2224 of the second tine 2206 can be relatively longer than the outer surface 2226 of the second tine 2206. As such, the distal end 2222 of the second tine 2206 can be angled from the inner surface 2224 to the outer surface 2226 and this angled portion can face outward relative to the surgical staple 2200.

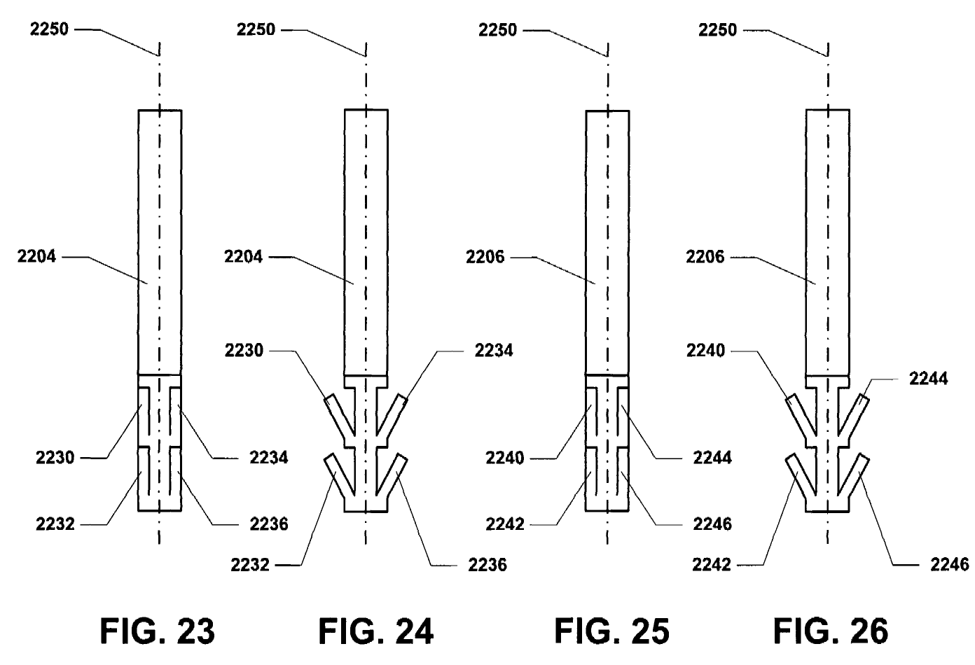

As illustrated in FIG. 23 and FIG. 24, the first tine 2204 can include a first tissue engagement structure 2230, a second tissue engagement structure 2232, a third tissue engagement structure 2234, and a fourth tissue engagement structure 2236 that can be incorporated into the distal end 2212 of the first tine 2204. Further, as shown in FIG. 25 and FIG. 26, the second tine 2206 can include a first tissue engagement structure 2240, a second tissue engagement structure 2242, a third tissue engagement structure 2244, and a fourth tissue engagement structure 2246 that can be incorporated into the distal end 2222 of the second tine 2206. As shown, the tissue engagement structures 2230, 2232, 2240, 2242 can be substantially straight arms that can be incorporated into the tines 2204, 2206.

The surgical staple 2200 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 2200 can be moved between a closed configuration and an open configuration or between an open configuration and a closed configuration, described above.

Further, the tissue engagement structures 2230, 2232, 2234, 2236, 2240, 2242, 2244, 2246 of each tine 2204, 2206 can be moved between a retracted configuration, shown in FIG. 23 and FIG. 24, and an extended configuration, shown in FIG. 25 and FIG. 26. In the retracted configuration, the tissue engagement structures 2230, 2232, 2234, 2236, 2240, 2242, 2244, 2246 can be substantially parallel to a longitudinal axis 2250 of the surgical staple 2200. Further, in the retracted configuration, the tissue engagement structures 2230, 2232, 2234, 2236, 2240, 2242, 2244, 2246 are substantially flush with the surfaces of the surgical staple 2200.

In the extended configuration, the tissue engagement structures 2230, 2232, 2234, 2236, 2240, 2242, 2244, 2246 can be angled with respect to the longitudinal axis 2250 of the surgical staple 2200. Further, in the extended configuration, each pair of adjacent tissue engagement structures can form a generally V-shaped structure that opens towards the base 2202 of the surgical staple 2200. Also, in the extended configuration the tissue engagement structures 2230, 2232, 2234, 2236, 2240, 2242, 2244, 2246 can engage tissue, e.g., bone or flesh, and substantially minimize the likelihood that the surgical staple 2200 will be prematurely withdrawn from the tissue.

In a particular embodiment, the tissue engagement structures 2230, 2232, 2234, 2236, 2240, 2242, 2244, 2246 of the surgical staple 2200 can be moved from the retracted configuration to the extended configuration by applying heat to the surgical staple 2200.

### Description of a Seventh Embodiment of a Two-Tine Surgical Staple

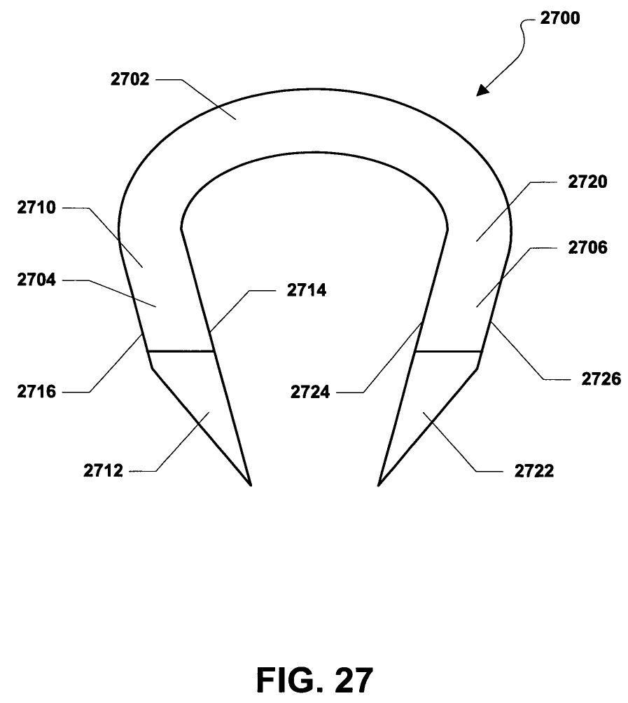

Referring to FIG. 27 through FIG. 31, a seventh embodiment of a two-tine surgical staple is shown and is generally designated 2700. As shown, the two-tine surgical staple 2700 can be generally U-shaped and can include a base 2702. A first tine 2704 and a second tine 2706 can extend from the base 2702.

As depicted, the first tine 2704 can include a proximal end 2710 and a distal end 2712. Moreover, the first tine 2704 can include an inner surface 2714 and an outer surface 2716. In a particular embodiment, the inner surface 2714 of the first tine 2704 can be relatively longer than the outer surface 2716 of the first tine 2704. As such, the distal end 2712 of the first tine 2704 can be angled from the inner surface 2714 to the outer surface 2716 and this angled portion can face outward relative to the surgical staple 2700.

The second tine 2706 can include a proximal end 2720 and a distal end 2722. Moreover, the second tine 2706 can include an inner surface 2724 and an outer surface 2726. In a particular embodiment, the inner surface 2724 of the second tine 2706 can be relatively longer than the outer surface 2726 of the second tine 2706. As such, the distal end 2722 of the second tine 2706 can be angled from the inner surface 2724 to the outer surface 2726 and this angled portion can face outward relative to the surgical staple 2700.

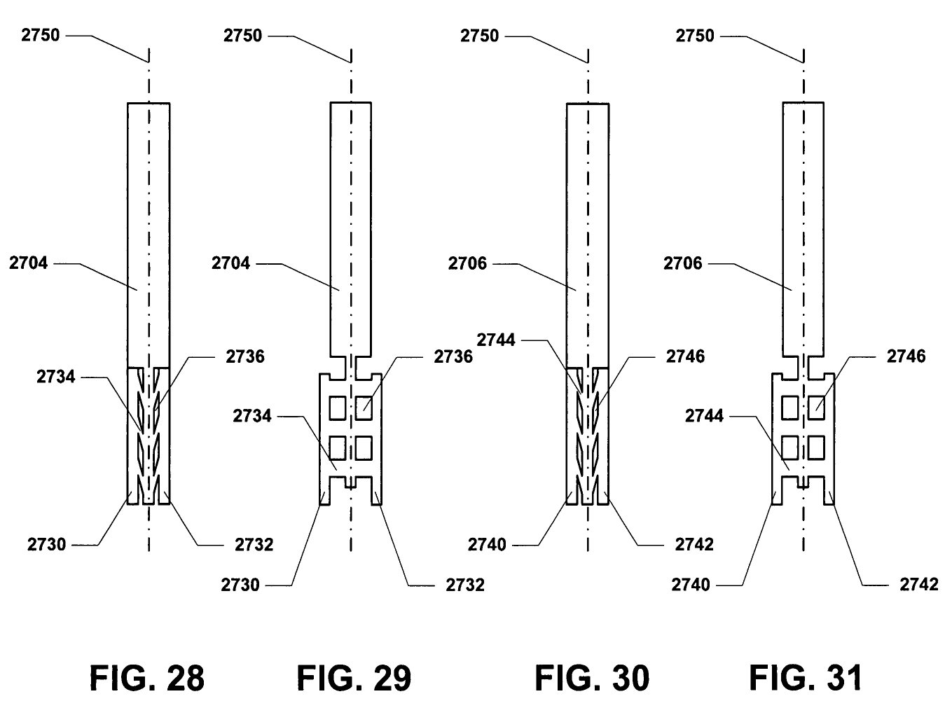

As illustrated in FIG. 28 and FIG. 29, the first tine 2704 can include a first tissue engagement structure 2730 and a second tissue engagement structure 2732. The first tissue engagement structure 2730 and the second tissue engagement structure 2732 can be coupled to a central structure 2734 by a plurality of arms 2736 that can be separated by a plurality of voids 2738 formed in the distal end 2712 of the first tine 2704. Further, as shown in FIG. 30 and FIG. 31, the second tine 2706 can include a first tissue engagement structure 2740 and a second tissue engagement structure 2742. The first tissue engagement structure 2740 and the second tissue engagement structure 2742 can be coupled to a central structure 2744 by a plurality of arms 2746 that can be separated by a plurality of voids 2748 formed in the distal end 2722 of the second tine 2706.

The surgical staple 2700 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 2700 can be moved between a closed configuration and an open configuration or between an open configuration and a closed configuration, described above.

Further, the tissue engagement structures 2730, 2732, 2740, 2742 of each tine 2704, 2706 can be moved between a retracted configuration, shown in FIG. 28 and FIG. 30, and an extended configuration, shown in FIG. 29 and FIG. 31. In the retracted configuration, the tissue engagement structures 2730, 2732, 2740, 2742 can be substantially parallel to a longitudinal axis 2750 of the surgical staple 2700. Further, in the retracted configuration, the tissue engagement structures 2730, 2732, 2740, 2742 are substantially flush with the outer surfaces of the surgical staple 2700. Also, in the retracted configuration, the arms 2736, 2746 that connect the tissue engagement structures 2730, 2732, 2740, 2742 to the central structures 2734, 2744 can be angled with respect to the longitudinal axis 2750 of the surgical staple 2700.

In the extended configuration, the tissue engagement structures 2730, 2732, 2740, 2742 can remain parallel to the longitudinal axis 2750 of the surgical staple 2700. However, in the extended configuration, the tissue engagement structures 2730, 2732, 2740, 2742 can extend beyond the outer surfaces of the surgical staple 2700. Also, in the extended configuration, the arms 2736, 2746 that connect the tissue engagement structures 2730, 2732, 2740, 2742 to the central structures 2734, 2744 can be substantially perpendicular to the longitudinal axis 2750 of the surgical staple 2700. In the extended configuration the tissue engagement structures 2730, 2732, 2740, 2742 can engage tissue, e.g., bone or flesh, and substantially minimize the likelihood that the surgical staple 2700 will be prematurely withdrawn from the tissue.

In a particular embodiment, the tissue engagement structures 2730, 2732, 2740, 2742 of the surgical staple 2700 can be moved from the retracted configuration to the extended configuration by applying heat to the surgical staple 2700.

### Description of an Eighth Embodiment of a Two-Tine Surgical Staple

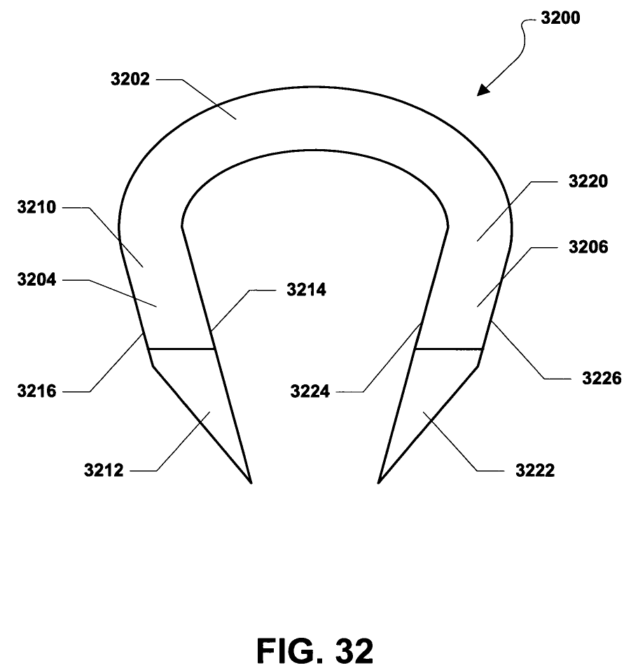

Referring to FIG. 32 through FIG. 36, an eighth embodiment of a two-tine surgical staple is shown and is generally designated 3200. As shown, the two-tine surgical staple 3200 can be generally U-shaped and can include a base 3202. A first tine 3204 and a second tine 3206 can extend from the base 3202.

As depicted, the first tine 3204 can include a proximal end 3210 and a distal end 3212. Moreover, the first tine 3204 can include an inner surface 3214 and an outer surface 3216. In a particular embodiment, the inner surface 3214 of the first tine 3204 can be relatively longer than the outer surface 3216 of the first tine 3204. As such, the distal end 3212 of the first tine 3204 can be angled from the inner surface 3214 to the outer surface 3216 and this angled portion can face outward relative to the surgical staple 3200.

The second tine 3206 can include a proximal end 3220 and a distal end 3222. Moreover, the second tine 3206 can include an inner surface 3224 and an outer surface 3226. In a particular embodiment, the inner surface 3224 of the second tine 3206 can be relatively longer than the outer surface 3226 of the second tine 3206. As such, the distal end 3222 of the second tine 3206 can be angled from the inner surface 3224 to the outer surface 3226 and this angled portion can face outward relative to the surgical staple 3200.

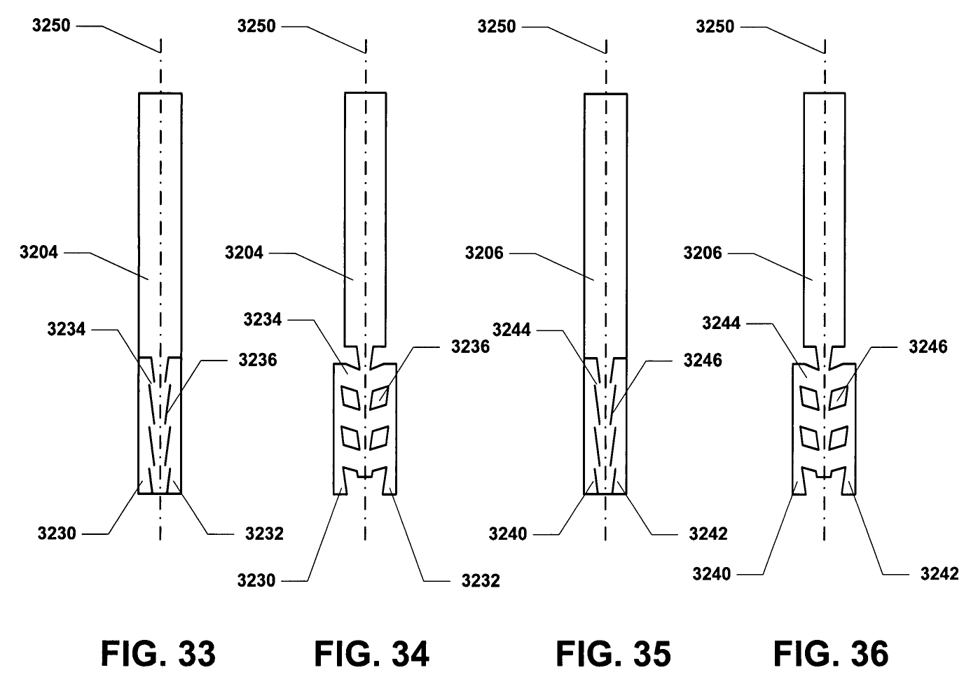

As illustrated in FIG. 33 and FIG. 34, the first tine 3204 can include a first tissue engagement structure 3230 and a second tissue engagement structure 3232. The first tissue engagement structure 3230 and the second tissue engagement structure 3232 can be coupled to a central structure 3234 by a plurality of arms 3236 that can be separated by a plurality of slits 3238 formed in the distal end 3212 of the first tine 3204. Further, as shown in FIG. 35 and FIG. 36, the second tine 3206 can include a first tissue engagement structure 3240 and a second tissue engagement structure 3242. The first tissue engagement structure 3240 and the second tissue engagement structure 3242 can be coupled to a central structure 3244 by a plurality of arms 3246 that can be separated by a plurality of slits 3248 formed in the distal end 3222 of the second tine 3206.

The surgical staple 3200 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 3200 can be moved between a closed configuration and an open configuration or between an open configuration and a closed configuration, described above.

Further, the tissue engagement structures 3230, 3232, 3240, 3242 of each tine 3204, 3206 can be moved between a retracted configuration, shown in FIG. 33 and FIG. 35, and an extended configuration, shown in FIG. 34 and FIG. 36. In the retracted configuration, the tissue engagement structures 3230, 3232, 3240, 3242 can be substantially parallel to a longitudinal axis 3250 of the surgical staple 3200. Further, in the retracted configuration, the tissue engagement structures 3230, 3232, 3240, 3242 are substantially flush with the outer surfaces of the surgical staple 3200.

In the extended configuration, the tissue engagement structures 3230, 3232, 3240, 3242 can remain parallel to the longitudinal axis 3250 of the surgical staple 3200. However, in the extended configuration, the tissue engagement structures 3230, 3232, 3240, 3242 can extend beyond the outer surfaces of the surgical staple 3200. Also, in the extended configuration, the slits 3238, 3248 that separate the arms 3236, 3246 that connect the tissue engagement structures 3230, 3232, 3240, 3242 to the central structures 3234, 3244 can open up and form a plurality of openings in the distal end 3212, 3222 of each tine 3204, 3206. In the extended configuration the tissue engagement structures 3230, 3232, 3240, 3242 can engage tissue, e.g., bone or flesh, and substantially minimize the likelihood that the surgical staple 3200 will be prematurely withdrawn from the tissue.

In a particular embodiment, the tissue engagement structures 3230, 3232, 3240, 3242 of the surgical staple 3200 can be moved from the retracted configuration to the extended configuration by applying heat to the surgical staple 3200.

### Description of a Ninth Embodiment of a Two-Tine Surgical Staple

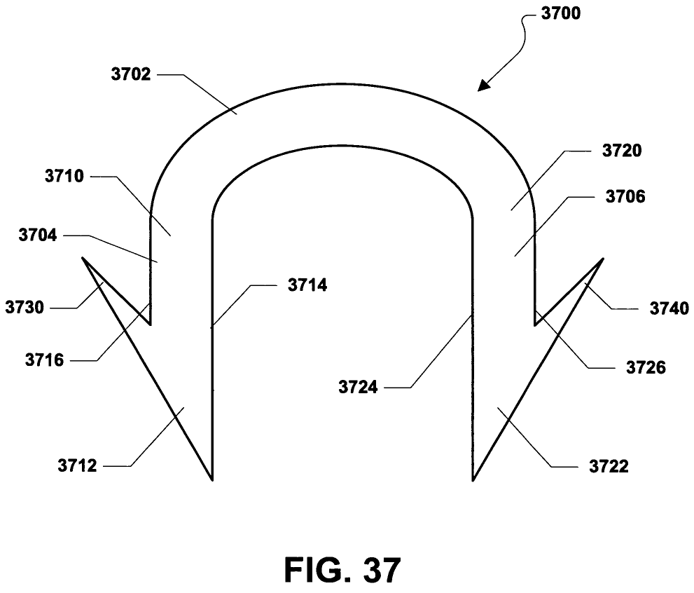

Referring to FIG. 37 through FIG. 41, a ninth embodiment of a two-tine surgical staple is shown and is generally designated 3700. As shown, the two-tine surgical staple 3700 can be generally U-shaped and can include a base 3702. A first tine 3704 and a second tine 3706 can extend from the base 3702.

As depicted, the first tine 3704 can include a proximal end 3710 and a distal end 3712. Moreover, the first tine 3704 can include an inner surface 3714 and an outer surface 3716. In a particular embodiment, the inner surface 3714 of the first tine 3704 can be relatively longer than the outer surface 3716 of the first tine 3704. As such, the distal end 3712 of the first tine 3704 can be angled from the inner surface 3714 to the outer surface 3716 and this angled portion can face outward relative to the surgical staple 3700.

The second tine 3706 can include a proximal end 3720 and a distal end 3722. Moreover, the second tine 3706 can include an inner surface 3724 and an outer surface 3726. In a particular embodiment, the inner surface 3724 of the second tine 3706 can be relatively longer than the outer surface 3726 of the second tine 3706. As such, the distal end 3722 of the second tine 3706 can be angled from the inner surface 3724 to the outer surface 3726 and this angled portion can face outward relative to the surgical staple 3700.

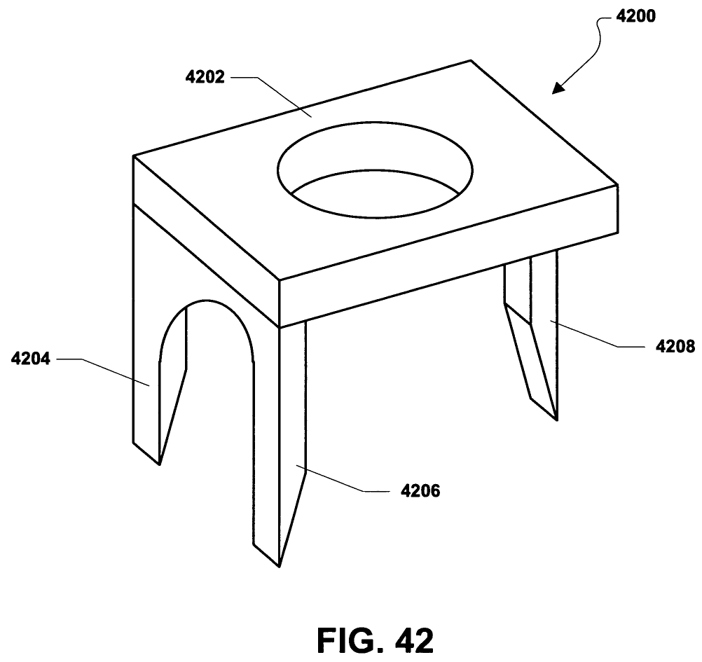

As illustrated in FIG. 37 through FIG. 39, the first tine 3704 can include a first barb 3730. The first barb 3730 can include a first tissue engagement structure 3732 and a second tissue engagement structure 3734. Further, as shown in FIG. 40 and FIG. 41, the second tine 3706 can include a second barb 3740. The second barb 3740 can include a first tissue engagement structure 3742 and a second tissue engagement structure 3744.

The surgical staple 3700 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. In a particular embodiment, the surgical staple 3700 can be moved between a closed configuration and an open configuration or between an open configuration and a closed configuration, described above.

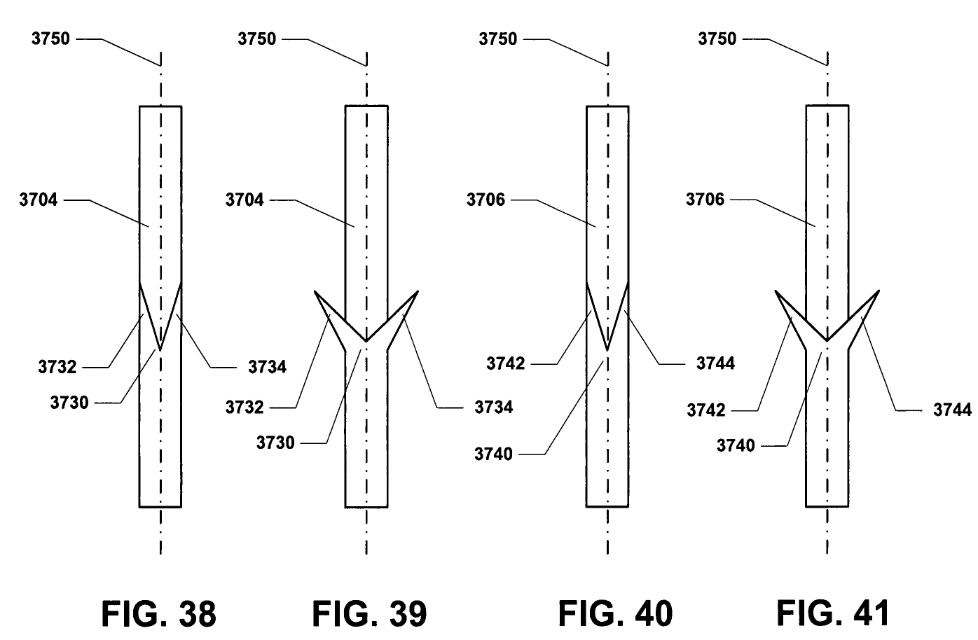

Further, the tissue engagement structures 3732, 3734, 3742, 3744 of each tine 3704, 3706 can be moved between a retracted configuration, shown in FIG. 38 and FIG. 40, and an extended configuration, shown in FIG. 39 and FIG. 41. In the retracted configuration, the tissue engagement structures 3732, 3734, 3742, 3744 can be substantially flush with the outer surfaces of the surgical staple 3700.

In the extended configuration, the tissue engagement structures 3732, 3734, 3742, 3744 can extend beyond the outer surfaces of the surgical staple 3700. Also, in the extended configuration, the tissue engagement structures 3732, 3734, 3742, 3744 can be angled with respect to a longitudinal axis 3750 of the surgical staple 3700. Further, in the extended configuration, each pair of adjacent tissue engagement structures can form a generally V-shaped structure that toward the base 3702 of the surgical staple 3700. In the extended configuration the tissue engagement structures 3732, 3734, 3742, 3744 can engage tissue, e.g., bone or flesh, and substantially minimize the likelihood that the surgical staple 3700 will be prematurely withdrawn from the tissue.

In a particular embodiment, the tissue engagement structures 3732, 3734, 3742, 3744 of the surgical staple 3700 can be moved from the retracted configuration to the extended configuration by applying heat to the surgical staple 3700.

### Description of a First Embodiment of a Three-Tine Surgical Staple

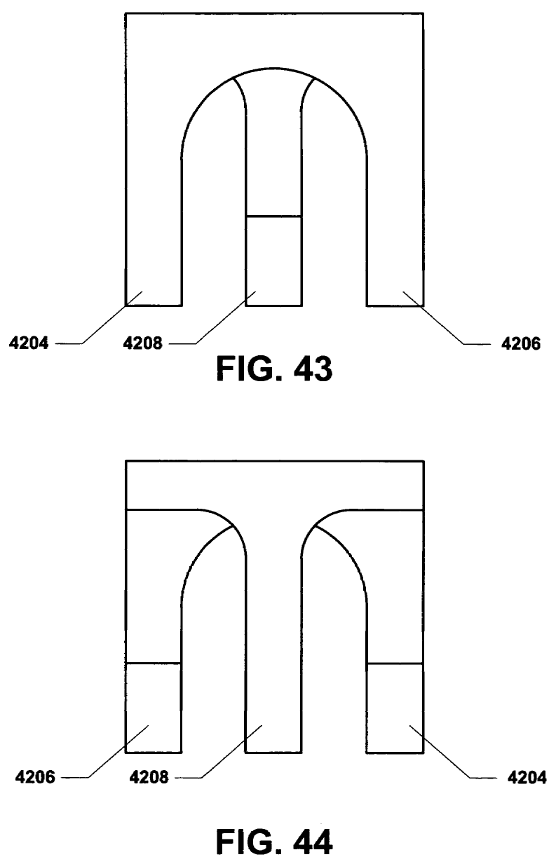

Referring initially to FIG. 42 through FIG. 48, a first embodiment of a three-tine surgical staple is shown and is generally designated 4200. As shown, the three-tine surgical staple 4200 can include a base 4202. A first tine 4204, a second tine 4206, and a third tine 4208 can extend from the base 4202. In a particular embodiment, the tines 4204, 4206, 4208 can extend substantially perpendicular from the base 4202.

As depicted, the first tine 4204 can include a proximal end 4210 and a distal end 4212. Moreover, the first tine 4204 can include an inner surface 4214 and an outer surface 4216. In a particular embodiment, the inner surface 4214 of the first tine 4204 can be relatively shorter than the outer surface 4216 of the first tine 4204. As such, the distal end 4212 of the first tine 4204 can be angled from the inner surface 4214 to the outer surface 4216 and this angled portion can face inward relative to the surgical staple 4200.

The second tine 4206 can include a proximal end 4220 and a distal end 4222. Moreover, the second tine 4206 can include an inner surface 4224 and an outer surface 4226. In a particular embodiment, the inner surface 4224 of the second tine 4206 can be relatively shorter than the outer surface 4226 of the second tine 4206. As such, the distal end 4222 of the second tine 4206 can be angled from the inner surface 4224 to the outer surface 4226 and this angled portion can face inward relative to the surgical staple 4200.

The third tine 4208 can include a proximal end 4230 and a distal end 4232. Moreover, the third tine 4208 can include an inner surface 4234 and an outer surface 4236. In a particular embodiment, the inner surface 4234 of the third tine 4208 can be relatively shorter than the outer surface 4236 of the third tine 4208. As such, the distal end 4232 of the third tine 4208 can be angled from the inner surface 4234 to the outer surface 4236 and this angled portion can face inward relative to the surgical staple 4200.

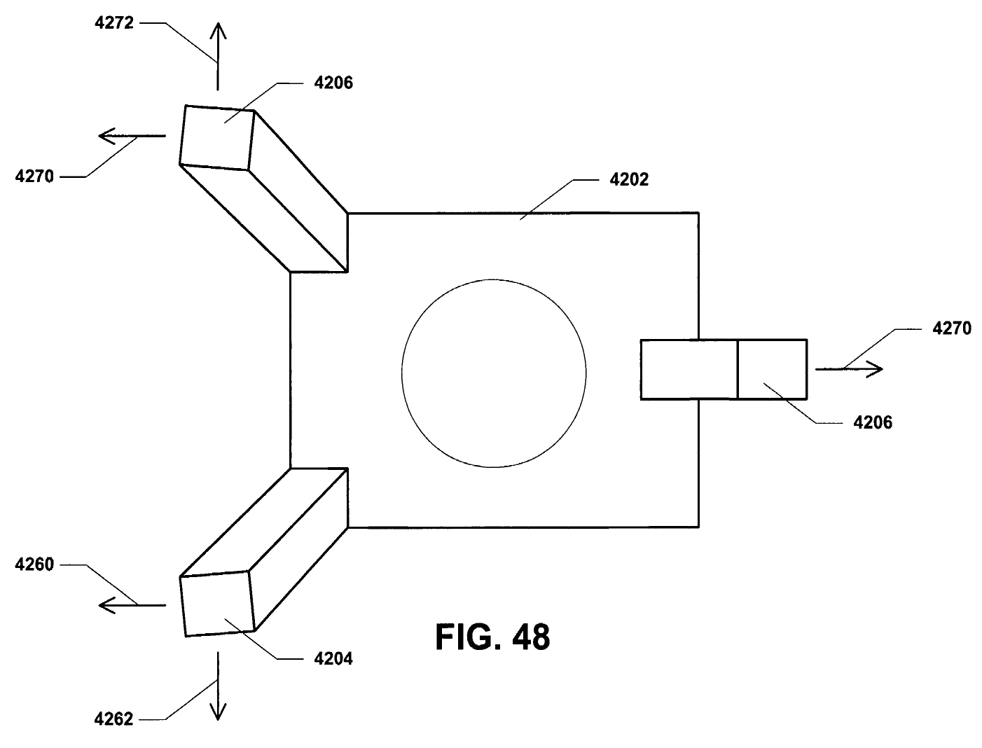

FIG. 42 and FIG. 48 indicate that the base 4202 of the surgical staple 4200 can be generally rectangular. Further, the base 4202 of the surgical staple 4200 can be formed with a hole 4240. In a particular embodiment, the hole 4240 can facilitate retrieval and insertion of the surgical staple 4200.

The surgical staple 4200 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol.

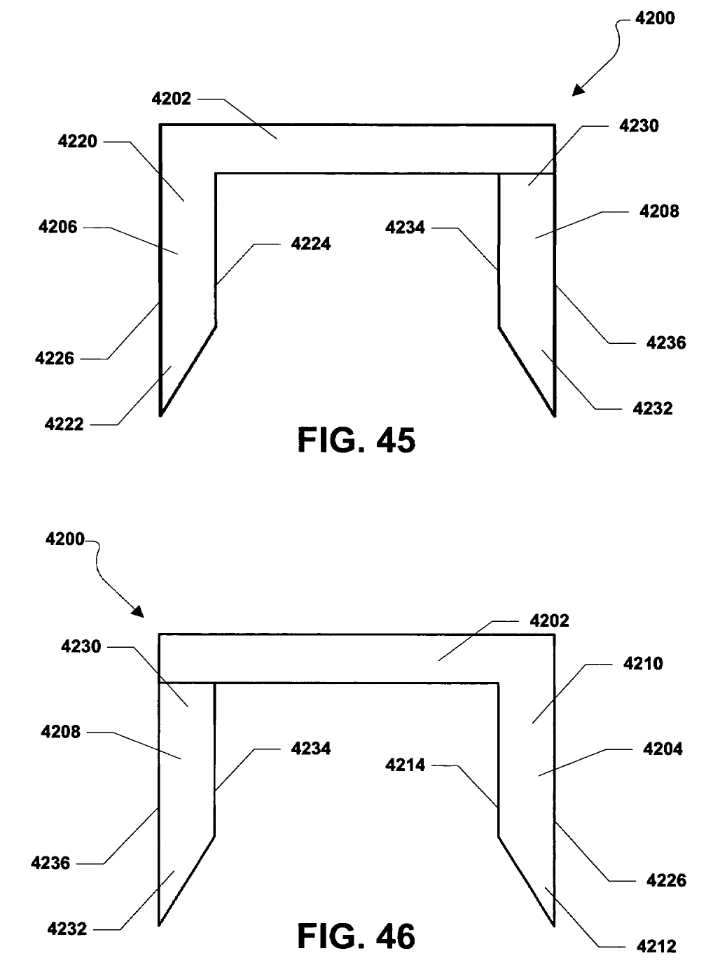

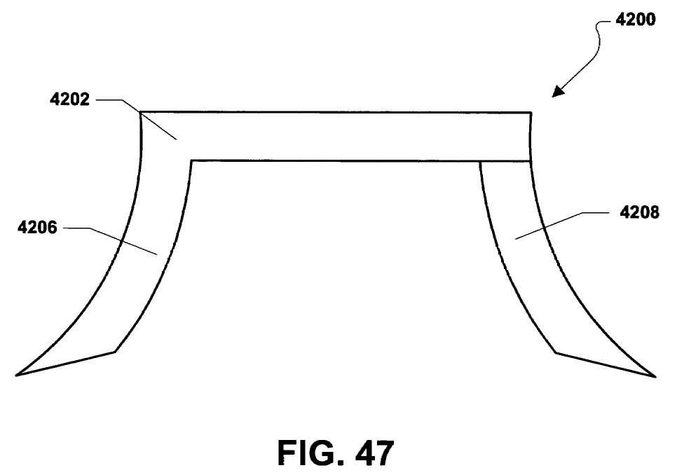

The surgical staple 4200 can be moved between a closed configuration, shown in FIG. 45 and FIG. 46, and an open configuration, shown in FIG. 47 and FIG. 48. In the closed configuration, the tines 4204, 4206, 4208 of the surgical staple 4200 can be substantially straight and substantially perpendicular to the base 4202 of the surgical staple 4200.

In the open configuration, the tines 4204, 4206, 4208 of the surgical staple 4200 can be bent outward relative to the base 4202 of the surgical staple 4200. In particular, the first tine 4204 can bend longitudinally with respect to the base 4202, as indicated by arrow 4260, and laterally with respect to the base 4202, as indicated by arrow 4262. Further, the second tine 4206 can bend longitudinally with respect to the base 4202, as indicated by arrow 4270, and laterally with respect to the base 4202, as indicated by arrow 4272. The third tine 4208 can move longitudinally with respect to the base 4202 as indicated by arrow 4280. Accordingly, the first tine 4204 and the second tine 4206 can bend outward relative to the third tine 4208 and the first tine 4204 and the second tine 4206 can be bend outward relative to each other.

In a particular embodiment, the surgical staple 4200 can be moved from the open configuration to the closed configuration by applying heat to the surgical staple 4200.

Further, in a particular embodiment, the first tine 4204, the second tine 4206, the third tine 4208, or a combination thereof can include one or more of the tissue engagement structures described herein.

### Description of a Second Embodiment of a Three-Tine Surgical Staple

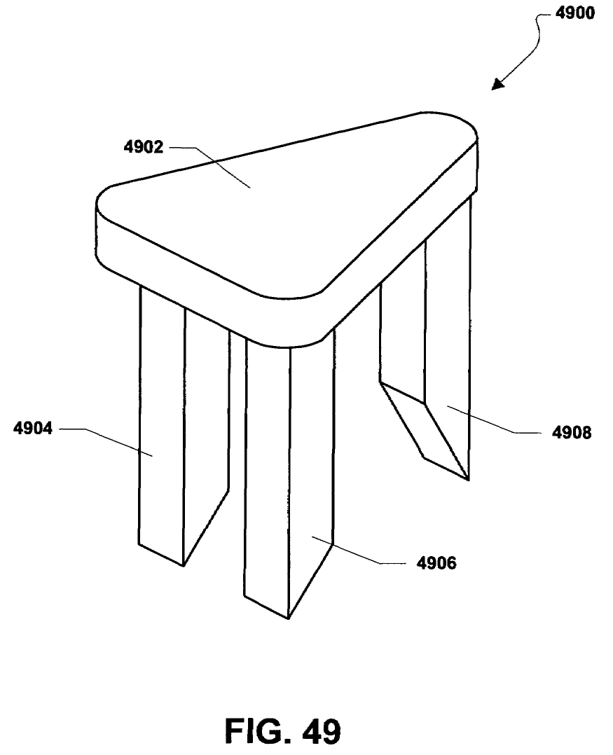

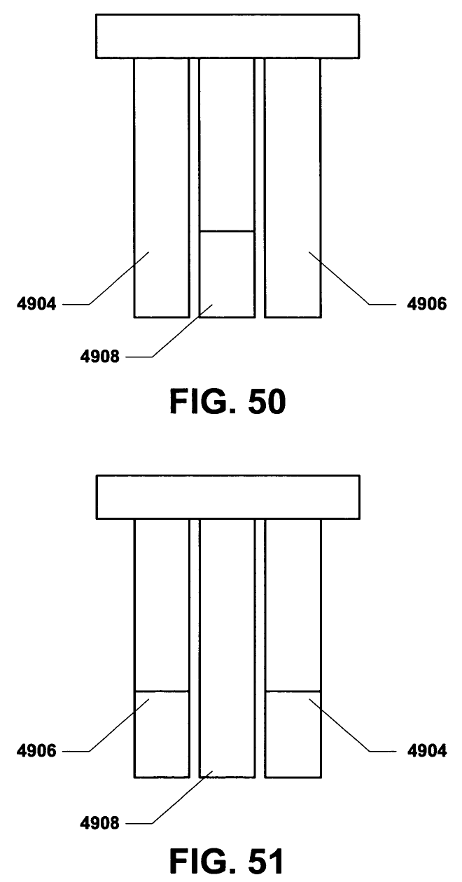

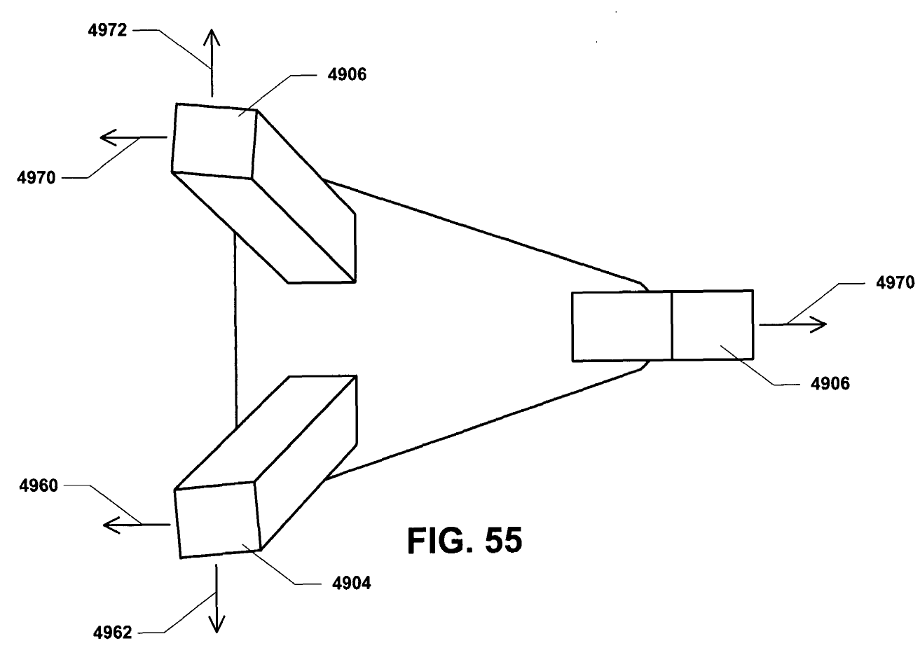

Referring initially to FIG. 49 through FIG. 55, a second embodiment of a three-tine surgical staple is shown and is generally designated 4900. As shown, the three-tine surgical staple 4900 can include a base 4902. In a particular embodiment, as illustrated in FIG. 49 and FIG. 55, the base 4902 of the surgical staple 4900 can be generally triangular. A first tine 4904, a second tine 4906, and a third tine 4908 can extend from the base 4902. For example, the first tine 4904 and the second tine 4906 can extend from one end of the base 4902 and the third tine 4908 can extend from the other end of the base 4902. In a particular embodiment, the tines 4904, 4906, 4908 can extend substantially perpendicular from the base 4902.

As depicted, the first tine 4904 can include a proximal end 4910 and a distal end 4912. Moreover, the first tine 4904 can include an inner surface 4914 and an outer surface 4916. In a particular embodiment, the inner surface 4914 of the first tine 4904 can be relatively shorter than the outer surface 4916 of the first tine 4904. As such, the distal end 4912 of the first tine 4904 can be angled from the inner surface 4914 to the outer surface 4916 and this angled portion can face inward relative to the surgical staple 4900.

The second tine 4906 can include a proximal end 4920 and a distal end 4922. Moreover, the second tine 4906 can include an inner surface 4924 and an outer surface 4926. In a particular embodiment, the inner surface 4924 of the second tine 4906 can be relatively shorter than the outer surface 4926 of the second tine 4906. As such, the distal end 4922 of the second tine 4906 can be angled from the inner surface 4924 to the outer surface 4926 and this angled portion can face inward relative to the surgical staple 4900.

The third tine 4908 can include a proximal end 4930 and a distal end 4932. Moreover, the third tine 4908 can include an inner surface 4934 and an outer surface 4936. In a particular embodiment, the inner surface 4934 of the third tine 4908 can be relatively shorter than the outer surface 4936 of the third tine 4908. As such, the distal end 4932 of the third tine 4908 can be angled from the inner surface 4934 to the outer surface 4936 and this angled portion can face inward relative to the surgical staple 4900.

The surgical staple 4900 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol.

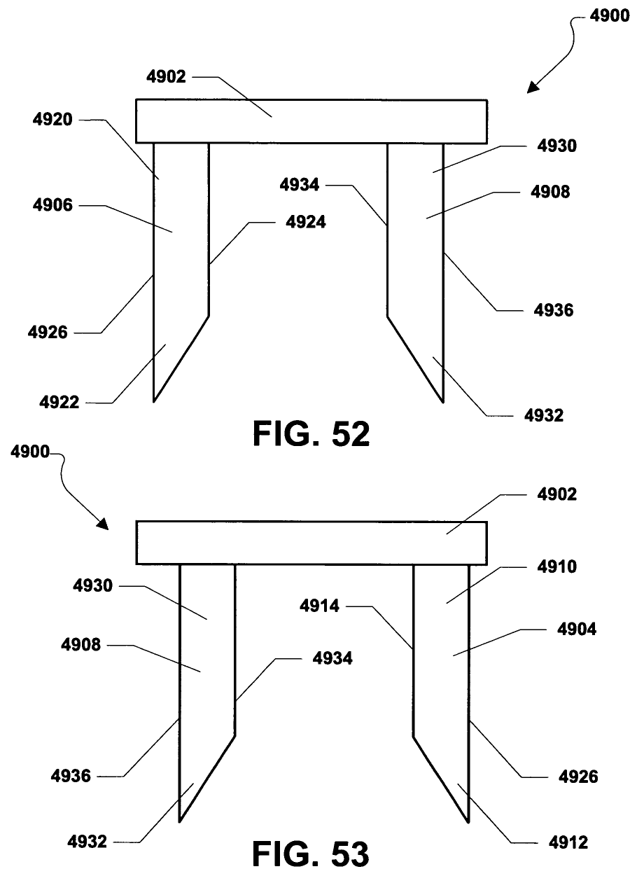

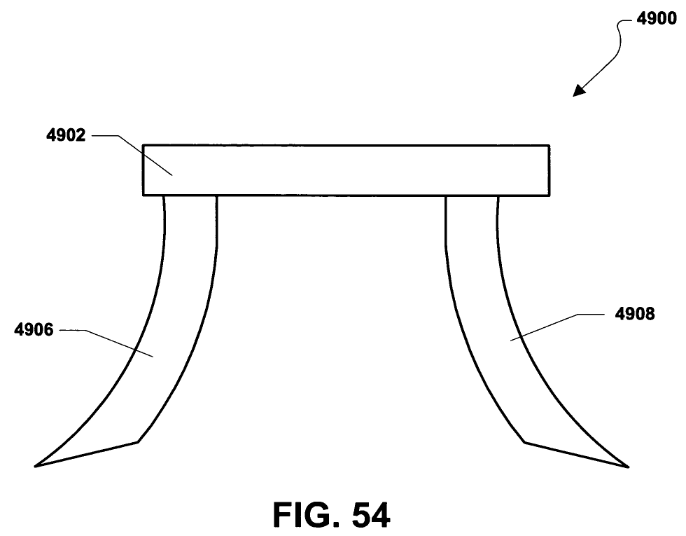

The surgical staple 4900 can be moved between a closed configuration, shown in FIG. 52 and FIG. 53, and an open configuration, shown in FIG. 54 and FIG. 55. In the closed configuration, the tines 4904, 4906, 4908 of the surgical staple 4900 can be substantially straight and substantially perpendicular to the base 4902 of the surgical staple 4900.

In the open configuration, the tines 4904, 4906, 4908 of the surgical staple 4900 can be bent outward relative to the base 4902 of the surgical staple 4900. In particular, the first tine 4904 can bend longitudinally with respect to the base 4902, as indicated by arrow 4960, and laterally with respect to the base 4902, as indicated by arrow 4962. Further, the second tine 4906 can bend longitudinally with respect to the base 4902, as indicated by arrow 4970, and laterally with respect to the base 4902, as indicated by arrow 4972. Accordingly, the first tine 4904 and the second tine 4906 can bend outward relative to the third tine 4908 and the first tine 4904 and the second tine 4906 can be bend outward relative to each other.

In a particular embodiment, the surgical staple 4900 can be moved from the open configuration to the closed configuration by applying heat to the surgical staple 4900.

Further, in a particular embodiment, the first tine 4904, the second tine 4906, the third tine 4908, or a combination thereof can include one or more of the tissue engagement structures described herein.

### Description of a First Embodiment of a Four-Tine Surgical Staple

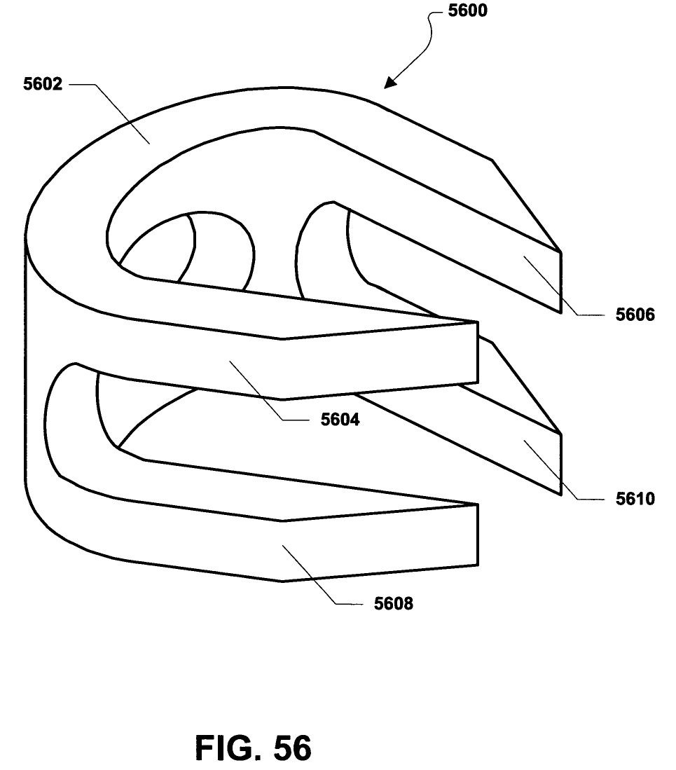

Referring initially to FIG. 56 through FIG. 59, a first embodiment of a four-tine surgical staple is shown and is generally designated 5600. As shown, the four-tine surgical staple 5600 can be generally U-shaped and can include a base 5602. A first tine 5604, a second tine 5606, a third tine 5608, and a fourth tine 5610 can extend from the base 5602.

As depicted, the first tine 5604 can include a proximal end 5620 and a distal end 5622. Moreover, the first tine 5604 can include an inner surface 5624 and an outer surface 5626. In a particular embodiment, the inner surface 5624 of the first tine 5604 can be relatively longer than the outer surface 5626 of the first tine 5604. As such, the distal end 5622 of the first tine 5604 can be angled from the inner surface 5624 to the outer surface 5626 and this angled portion can face outward relative to the surgical staple 5600.

The second tine 5606 can include a proximal end 5630 and a distal end 5632. Moreover, the second tine 5606 can include an inner surface 5634 and an outer surface 5636. In a particular embodiment, the inner surface 5634 of the second tine 5606 can be relatively longer than the outer surface 5636 of the second tine 5606. As such, the distal end 5632 of the second tine 5606 can be angled from the inner surface 5634 to the outer surface 5636 and this angled portion can face outward relative to the surgical staple 5600.

FIG. 56 also indicates that the base 5602 of the surgical staple 5600 can be formed with a hole 5660. In a particular embodiment, the hole 5660 can facilitate retrieval and insertion of the surgical staple 5600.

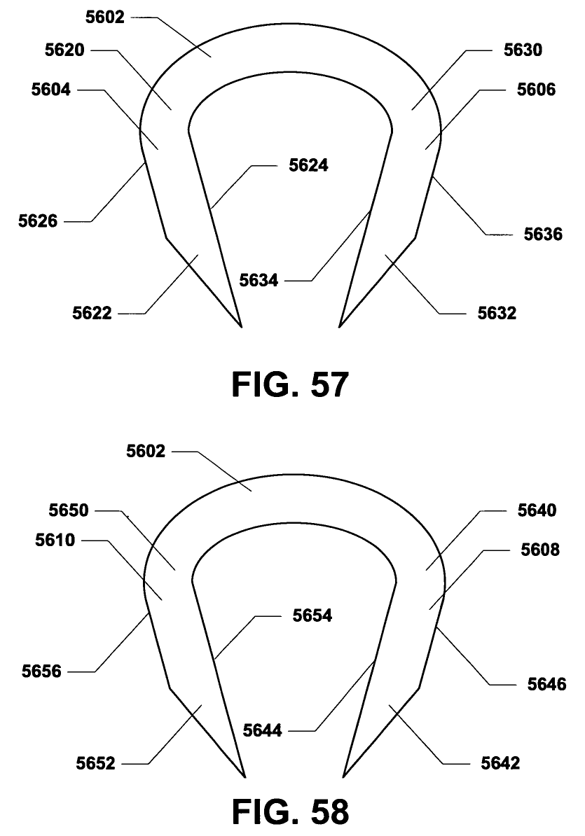

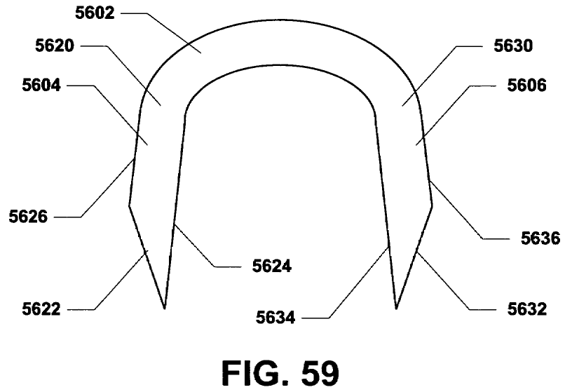

The surgical staple 5600 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol. The surgical staple 5600 can be moved between a closed configuration, shown in FIG. 57 and FIG. 58, and an open configuration, shown in FIG. 59. In a particular embodiment, the surgical staple 5600 can be moved from the closed configuration to the open configuration by applying heat to the surgical staple 5600.

Further, in a particular embodiment, the first tine 5604, the second tine 5606, the third tine 5608, the fourth tine 5610, or a combination thereof can include one or more of the tissue engagement structures described herein.

### Description of a Second Embodiment of a Four-Tine Surgical Staple

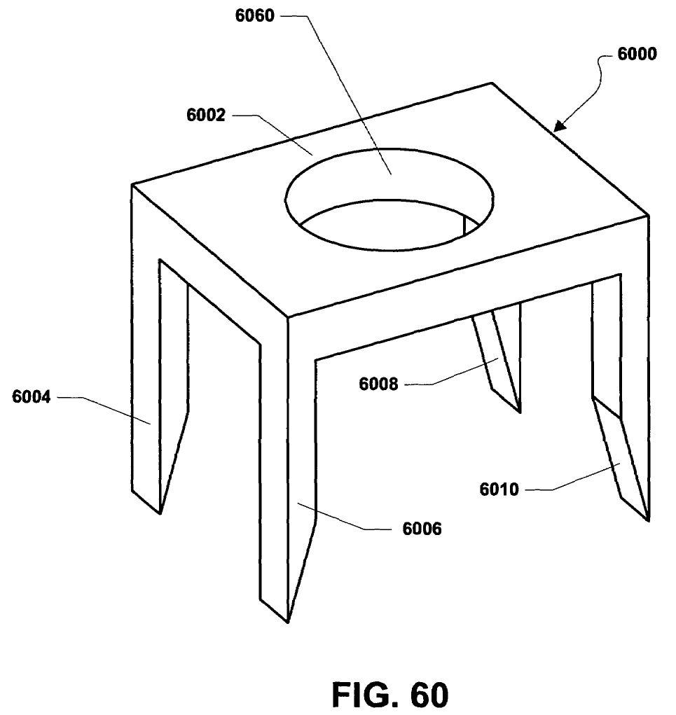

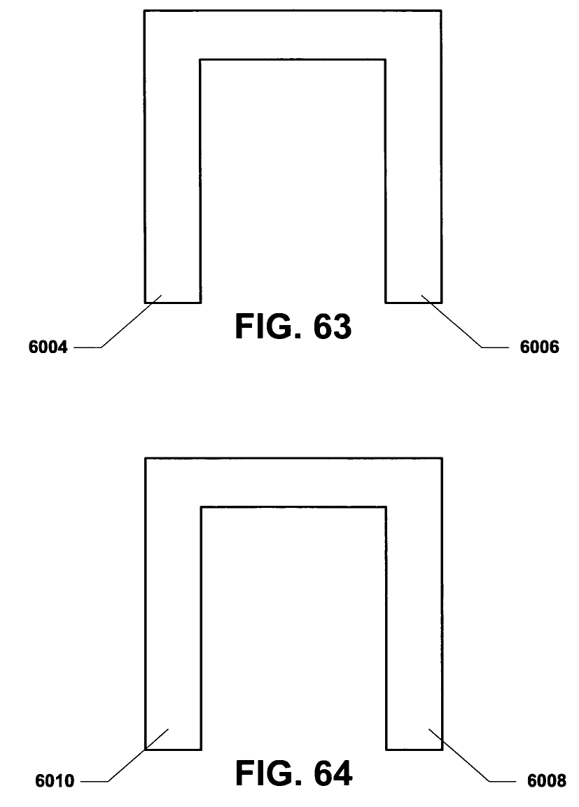

Referring initially to FIG. 60 through FIG. 66, a second embodiment of a four-tine surgical staple is shown and is generally designated 6000. As shown, the four-tine surgical staple 6000 can include a base 6002. A first tine 6004, a second tine 6006, a third tine 6008, and a fourth tine 6010 can extend from the base 6002. For example, the first tine 6004 and the second tine 6006 can extend from one end of the base 6002 and the third tine 6008 and the fourth tine 6010 can extend from the other end of the base 6002. In a particular embodiment, the tines 6004, 6006, 6008, 6010 can extend substantially perpendicular from the base 6002.

As depicted, the first tine 6004 can include a proximal end 6020 and a distal end 6022. Moreover, the first tine 6004 can include an inner surface 6024 and an outer surface 6026. In a particular embodiment, the inner surface 6024 of the first tine 6004 can be relatively shorter than the outer surface 6026 of the first tine 6004. As such, the distal end 6022 of the first tine 6004 can be angled from the inner surface 6024 to the outer surface 6026 and this angled portion can face inward relative to the surgical staple 6000.

The second tine 6006 can include a proximal end 6030 and a distal end 6032. Moreover, the second tine 6006 can include an inner surface 6034 and an outer surface 6036. In a particular embodiment, the inner surface 6034 of the second tine 6006 can be relatively shorter than the outer surface 6036 of the second tine 6006. As such, the distal end 6032 of the second tine 6006 can be angled from the inner surface 6034 to the outer surface 6036 and this angled portion can face inward relative to the surgical staple 6000.

The third tine 6008 can include a proximal end 6040 and a distal end 6042. Moreover, the third tine 6008 can include an inner surface 6044 and an outer surface 6046. In a particular embodiment, the inner surface 6044 of the third tine 6008 can be relatively shorter than the outer surface 6046 of the third tine 6008. As such, the distal end 6042 of the third tine 6008 can be angled from the inner surface 6044 to the outer surface 6046 and this angled portion can face inward relative to the surgical staple 6000.

The fourth tine 6010 can include a proximal end 6050 and a distal end 6052. Moreover, the fourth tine 6010 can include an inner surface 6054 and an outer surface 6056. In a particular embodiment, the inner surface 6054 of the fourth tine 6010 can be relatively shorter than the outer surface 6056 of the fourth tine 6010. As such, the distal end 6052 of the fourth tine 6010 can be angled from the inner surface 6054 to the outer surface 6056 and this angled portion can face inward relative to the surgical staple 6000.

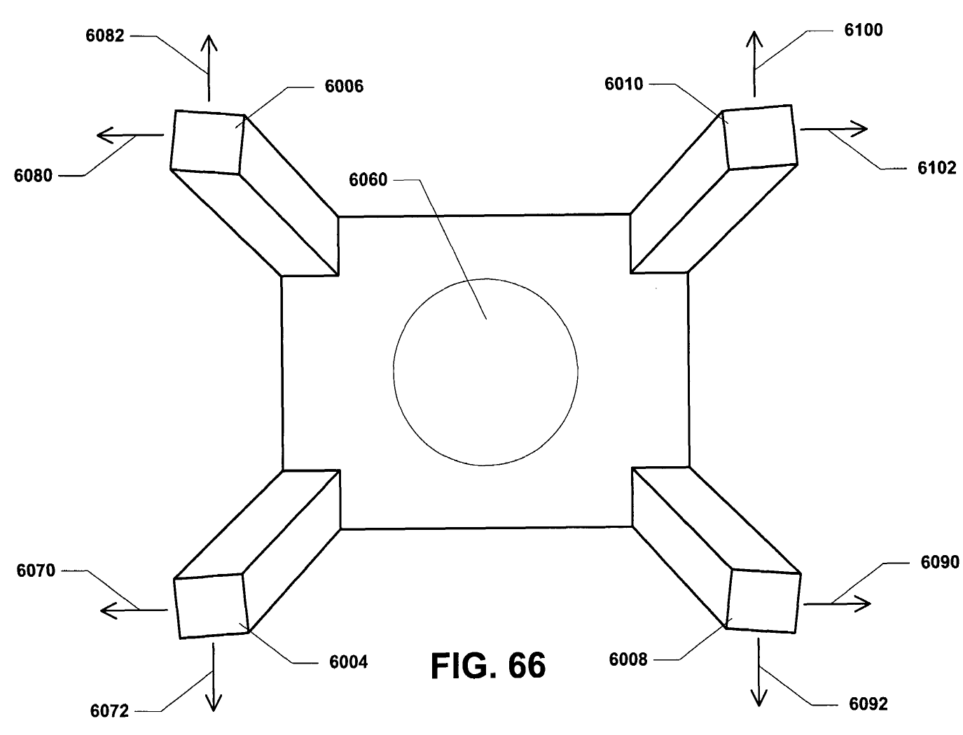

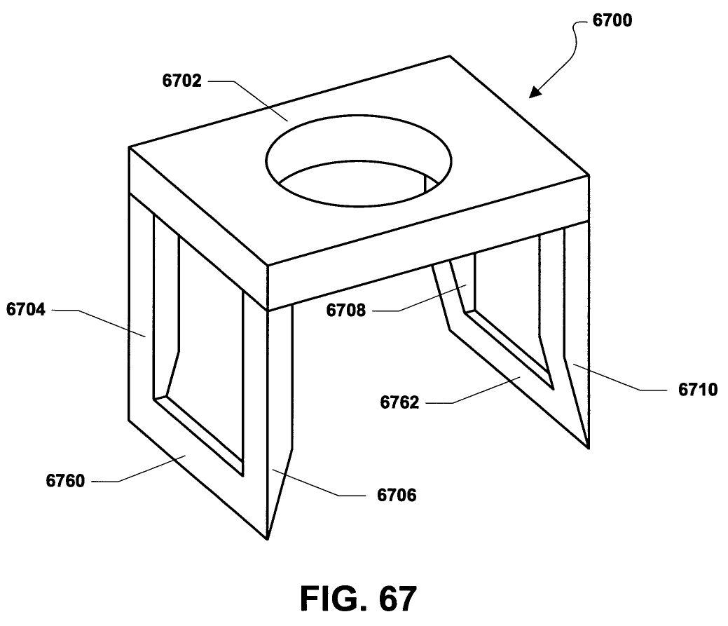

FIG. 60 and FIG. 66 indicate that the base 6002 of the surgical staple 6000 can be generally rectangular. Further, the base 6002 of the surgical staple 6000 can be formed with a hole 6060. In a particular embodiment, the hole 6060 can facilitate retrieval and insertion of the surgical staple 6000.

The surgical staple 6000 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol.

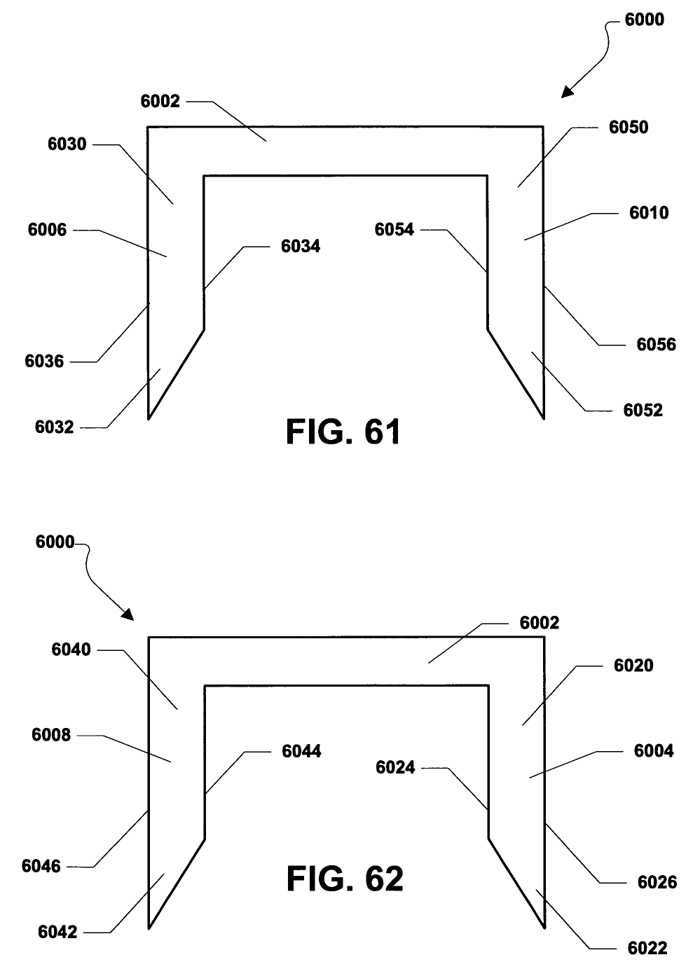

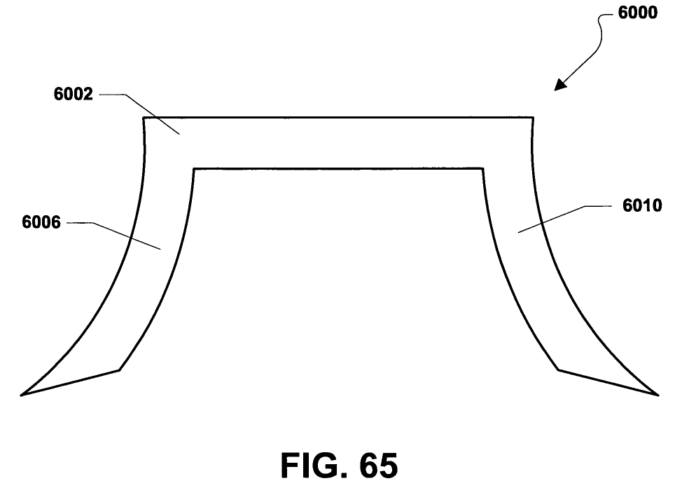

The surgical staple 6000 can be moved between a closed configuration, shown in FIG. 61 through FIG. 64, and an open configuration, shown in FIG. 65 and FIG. 66. In the closed configuration, the tines 6004, 6006, 6008, 6010 of the surgical staple 6000 can be substantially straight and substantially perpendicular to the base 6002 of the surgical staple 6000.

In the open configuration, the tines 6004, 6006, 6008, 6010 of the surgical staple 6000 can be bent outward relative to the base 6002 of the surgical staple 6000. In particular, the first tine 6004 can bend longitudinally with respect to the base 6002, as indicated by arrow 6070, and laterally with respect to the base 6002, as indicated by arrow 6072. Further, the second tine 6006 can bend longitudinally with respect to the base 6002, as indicated by arrow 6080, and laterally with respect to the base 6002, as indicated by arrow 6082. The third tine 6008 can bend longitudinally with respect to the base 6002, as indicated by arrow 6090, and laterally with respect to the base 6002, as indicated by arrow 6092. Also, the fourth tine 6010 can bend longitudinally with respect to the base 6002, as indicated by arrow 6100, and laterally with respect to the base 6002, as indicated by arrow 6102.

Accordingly, the first tine 6004 and the second tine 6006 can bend outward relative to the third tine 6008 and the fourth tine 6010. Moreover, the first tine 6004 and the second tine 6006 can be bend outward relative to each other and the third tine 6008 and the fourth tine 6010 can bend outward relative to each other.

In a particular embodiment, the surgical staple 6000 can be moved from the open configuration to the closed configuration by applying heat to the surgical staple 6000.

Further, in a particular embodiment, the first tine 6004, the second tine 6006, the third tine 6008, the fourth tine 6010, or a combination thereof can include one or more of the tissue engagement structures described herein.

### Description of a Third Embodiment of a Four-Tine Surgical Staple

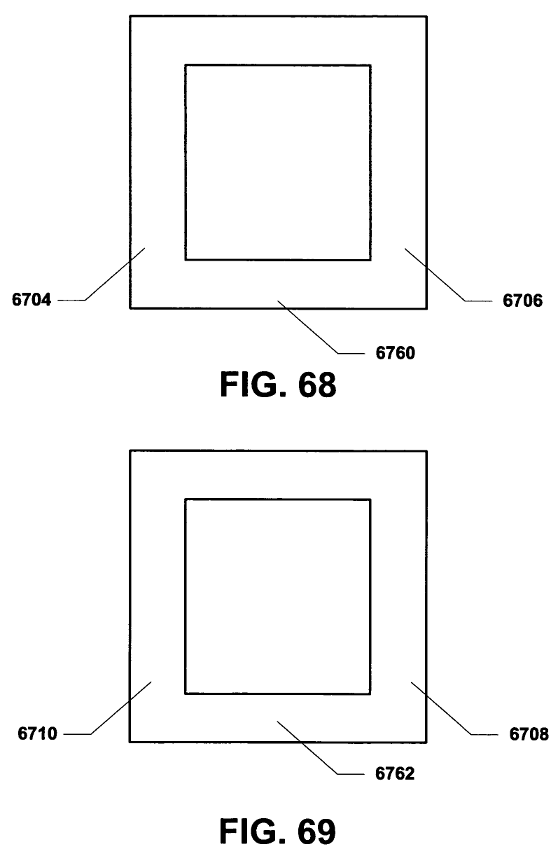

Referring initially to FIG. 67 through FIG. 72, a third embodiment of a four-tine surgical staple is shown and is generally designated 6700. As shown, the four-tine surgical staple 6700 can include a base 6702. A first tine 6704, a second tine 6706, a third tine 6708, and a fourth tine 6710 can extend from the base 6702. For example, the first tine 6704 and the second tine 6706 can extend from one end of the base 6702 and the third tine 6708 and the fourth tine 6710 can extend from the other end of the base 6702. In a particular embodiment, the tines 6704, 6706, 6708, 6710 can extend substantially perpendicular from the base 6702.

As depicted, the first tine 6704 can include a proximal end 6720 and a distal end 6722. Moreover, the first tine 6704 can include an inner surface 6724 and an outer surface 6726. In a particular embodiment, the inner surface 6724 of the first tine 6704 can be relatively shorter than the outer surface 6726 of the first tine 6704. As such, the distal end 6722 of the first tine 6704 can be angled from the inner surface 6724 to the outer surface 6726 and this angled portion can face inward relative to the surgical staple 6700.

The second tine 6706 can include a proximal end 6730 and a distal end 6732. Moreover, the second tine 6706 can include an inner surface 6734 and an outer surface 6736. In a particular embodiment, the inner surface 6734 of the second tine 6706 can be relatively shorter than the outer surface 6736 of the second tine 6706. As such, the distal end 6732 of the second tine 6706 can be angled from the inner surface 6734 to the outer surface 6736 and this angled portion can face inward relative to the surgical staple 6700.

The third tine 6708 can include a proximal end 6740 and a distal end 6742. Moreover, the third tine 6708 can include an inner surface 6744 and an outer surface 6746. In a particular embodiment, the inner surface 6744 of the third tine 6708 can be relatively shorter than the outer surface 6746 of the third tine 6708. As such, the distal end 6742 of the third tine 6708 can be angled from the inner surface 6744 to the outer surface 6746 and this angled portion can face inward relative to the surgical staple 6700.

The fourth tine 6710 can include a proximal end 6750 and a distal end 6752. Moreover, the fourth tine 6710 can include an inner surface 6754 and an outer surface 6756. In a particular embodiment, the inner surface 6754 of the fourth tine 6710 can be relatively shorter than the outer surface 6756 of the fourth tine 6710. As such, the distal end 6752 of the fourth tine 6710 can be angled from the inner surface 6754 to the outer surface 6756 and this angled portion can face inward relative to the surgical staple 6700.

In a particular embodiment, a first arm 6760 can connect the distal end 6722 of the first tine 6704 to the distal end 6732 of the second tine 6706. Further, a second arm 6762 can connect the distal end 6742 of the third tine 6708 to the distal end 6752 of the fourth tine 6710.

FIG. 67 indicates that the base 6702 of the surgical staple 6700 can be generally rectangular. Further, the base 6702 of the surgical staple 6700 can be formed with a hole 6750. In a particular embodiment, the hole 6750 can facilitate retrieval and insertion of the surgical staple 6700.

The surgical staple 6700 can be made from a metal; a metal alloy, such as a biocompatible, shape memory metal alloy; a biocompatible, shape memory polymer; or a combination thereof. For example, the shape memory metal alloy can be a metal alloy of titanium and nickel, e.g., nitinol.

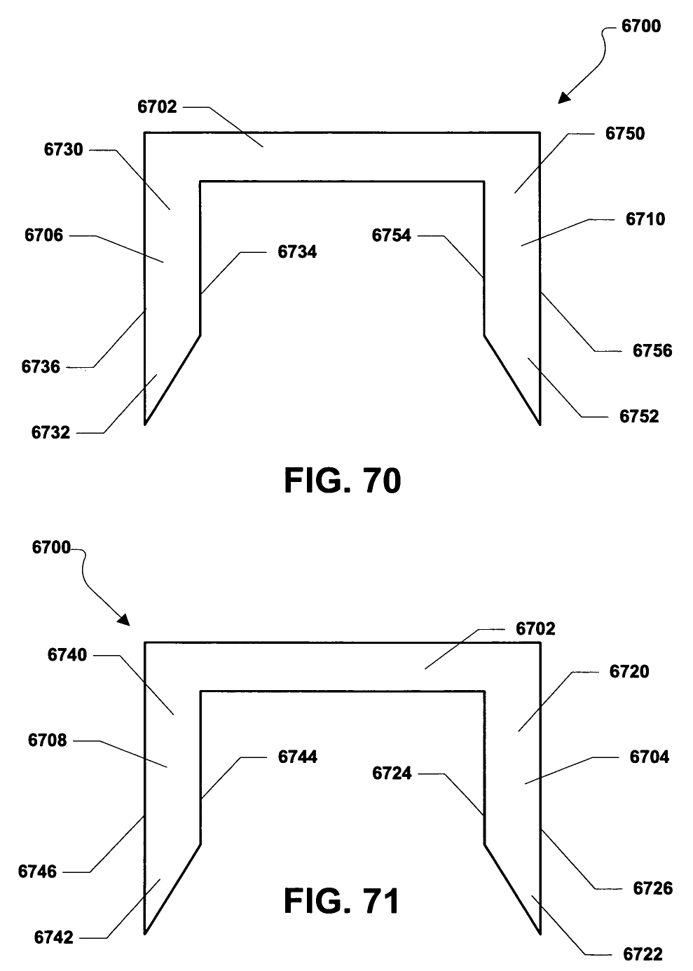

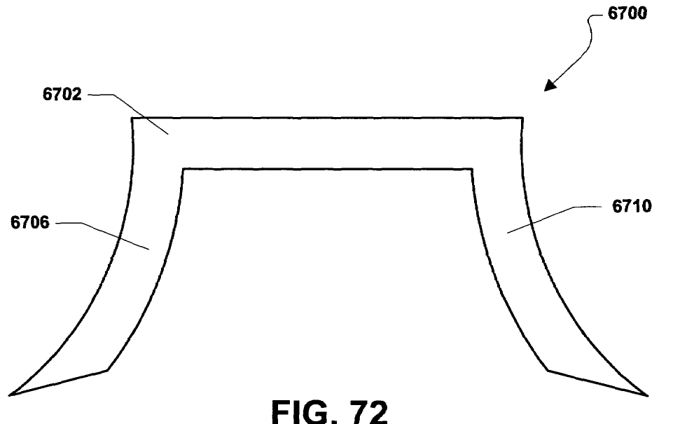

The surgical staple 6700 can be moved between a closed configuration, shown in FIG. 70 through FIG. 71, and an open configuration, shown in FIG. 72. In the closed configuration, the tines 6704, 6706, 6708, 6710 of the surgical staple 6700 can be substantially straight and substantially perpendicular to the base 6702 of the surgical staple 6700.

In the open configuration, the tines 6704, 6706, 6708, 6710 of the surgical staple 6700 can be bent outward relative to the base 6702 of the surgical staple 6700. In particular, the first tine 6704 and the second tine 6706 can bend outward relative to the third tine 6708 and the fourth tine 6710.

In a particular embodiment, the surgical staple 6700 can be moved from the open configuration to the closed configuration by applying heat to the surgical staple 6700.

Further, in a particular embodiment, the first tine 6704, the second tine 6706, the third tine 6708, the fourth tine 6710, or a combination thereof can include one or more of the tissue engagement structures described herein.

### CONCLUSION

With the configuration of structure described above, the surgical staple provides a device that can be used to close a wound, e.g., a flesh wound. Further, the surgical staple provides a device that can be used to repair a damaged bone. Also, the surgical staple can be used to treat a spinal deformity. A surgical staple according to one or more of the embodiments herein can include one or more of the various features described herein. Further, a surgical staple according to one or more of the embodiments herein can include any combination of the various features described herein.

In one or more of the embodiments described herein, the tines are angled inward or outward relative to the surgical staple. The angled tines can assist in opening or closing the surgical staple as the surgical staple is inserted in flesh or bone. Further, the angled tines can assist in moving the surgical staple in the direction of a memory shape associated with the surgical staple. Also, in one or more of the embodiments described herein, one or more barbs may be moved from an extended configuration, i.e., a memory configuration or memory shape, to a retracted configuration during insertion. Upon warming within the tissue, or other application of heat, the one or more barbs may then return to the extended configuration, i.e., the memory shape.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. A surgical staple, comprising:
a base;
at least one tine extending from the base;
at least one tissue engagement structure incorporated into the at least one tine, wherein the at least one tissue engagement structure is movable between a retracted configuration and an extended configuration.

2. The surgical staple of claim 1, wherein the at least one tine includes a proximal end and a distal end and wherein the at least one tissue engagement structure comprises a first tissue engagement structure and a second tissue engagement structure formed in the distal end of the at least one tine.

3. The surgical staple of claim 2, wherein the first tissue engagement structure comprises a substantially straight arm and the second tissue engagement structure comprises a substantially straight arm.

4. The surgical staple of claim 3, wherein the first tissue engagement structure is separated from the second tissue engagement structure by a void formed in the distal end of the at least one tine.

5. The surgical staple of claim 4, wherein the first tissue engagement structure and the second tissue engagement structure are substantially parallel to a longitudinal axis of the surgical staple in the retracted configuration.

6. The surgical staple of claim 5, wherein the first tissue engagement structure and the second tissue engagement structure are angled with respect to the longitudinal axis in the extended configuration.

7. The surgical staple of claim 6, wherein the first tissue engagement structure and the second tissue engagement structure form a generally V-shaped structure in the extended configuration.

8. The surgical staple of claim 7, wherein the generally V-shaped structure opens away from the base in the extended configuration.

9. The surgical staple of claim 4, wherein the first tissue engagement structure and the second tissue engagement structure are bowed with respect to the longitudinal axis in the extended configuration.

10. The surgical staple of claim 9, wherein the first tissue engagement structure and the second tissue engagement structure form a generally elliptical structure in the extended configuration.

11. The surgical staple of claim 3, wherein the first tissue engagement structure is separated from the second tissue engagement structure by a slit formed in the distal end of the at least one tine.

12. The surgical staple of claim 1 1, wherein the first tissue engagement structure and the second tissue engagement structure are substantially parallel to a longitudinal axis of the surgical staple in the retracted configuration.

13. The surgical staple of claim 12, wherein the first tissue engagement structure is folded at or near a midpoint in the extended configuration and the second tissue engagement structure is folded at or near a midpoint in the extended configuration.

14. The surgical staple of claim 13, wherein the first tissue engagement structure and the second tissue engagement structure form a generally diamond-shaped structure in the extended configuration.

15. The surgical staple of claim 14, wherein the slit opens and forms a generally diamond-shaped opening in the extended configuration.

16. The surgical staple of claim 1, wherein the at least one tine includes a proximal end and a distal end and wherein the at least one tissue engagement structure comprises a first tissue engagement structure, a second tissue engagement structure, a third tissue engagement structure, and a fourth tissue engagement structure formed in the distal end of the at least one tine.

17-23. (canceled)

24. The surgical staple of claim 3, wherein the distal end of the at least one tine includes a central structure and the first tissue engagement structure is coupled to the central structure by a first plurality of arms.

25. The surgical staple of claim 24, wherein the second tissue engagement structure is coupled to the central structure by a second plurality of arms.

26. The surgical staple of claim 25, wherein the first plurality of arms are separated by a first plurality of voids.

27. The surgical staple of claim 26, wherein the second plurality of arms are separated by a second plurality of voids.

28. The surgical staple of claim 27, wherein the first tissue engagement structure and the second tissue engagement structure are substantially parallel to a longitudinal axis of the surgical staple in the retracted configuration.

29. The surgical staple of claim 28, wherein the first tissue engagement structure and the second tissue engagement structure are substantially flush with a first outer surface and a second outer surface of the surgical staple in the retracted configuration.

30. The surgical staple of claim 29, wherein the first plurality of arms and the second plurality of arms are angled with respect to the longitudinal axis in the retracted configuration.

31. The surgical staple of claim 30, wherein the first tissue engagement structure and the second tissue engagement structure extend beyond the first outer surface and the second outer surface of the surgical staple in the extended configuration.

32. The surgical staple of claim 31, wherein the first plurality of arms and the second plurality of arms are substantially perpendicular to the longitudinal axis in the extended configuration.

33-45. (canceled)

46. The surgical staple of claim 2, wherein the distal end of the at least one tine includes an angled portion and wherein the angled portion of the tine moves the tine inward or outward as the surgical staple is driven into tissue.

47. The surgical staple of claim 46 wherein the angled portion of the at least one time moves the tine toward a memory shape as the surgical staple is driven into tissue.

48. The surgical staple of claim 1, wherein the at least one tissue engagement structure moves to a retracted configuration during insertion and returns to the extended configuration when heat is applied to the surgical staple.

49. A surgical staple, comprising:
a base;
a first tine extending from the base;
a first tissue engagement structure incorporated into the first tine;
a second tissue engagement structure incorporated into the first tine;
a second tine extending from the base opposite the first tine;
a first tissue engagement structure incorporated into the second tine; and
a second tissue engagement structure incorporated into the second tine, wherein each tissue engagement structure is movable between a retracted configuration and an extended configuration.

50-60. (canceled)

61. The surgical staple of any of the preceding claims, wherein at least a portion of the staple comprises a shape memory material.

62. The surgical staple of claim 61, wherein the shape memory material is nitinol.
